(19) United States
(12) Patent Application Publication
(10) Pub. No.: US 2024/0003884 A1
(43) Pub. Date: Jan. 4, 2024

(54) METHODS AND COMPOSITIONS USING NON-INVASIVE PLASMA GLYCOMIC AND METABOLIC BIOMARKERS OF POST-TREATMENT CONTROL OF HIV

(71) Applicant: The Wistar Institute of Anatomy and Biology, Philadelphia, PA (US)

(72) Inventor: Mohamed Abdel-Mohsen, Wynnewood, PA (US)

(21) Appl. No.: 18/251,739

(22) PCT Filed: Nov. 9, 2021
(86) PCT No.: PCT/US2021/058607

§ 371 Date: May 4, 2023

Related U.S. Application Data
(60) Provisional application No. 63/112,047, filed on Nov. 10, 2020.

(51) Int. Cl. G01N 33/569

(52) U.S. Cl. CPC G01N 33/56988; G01N 2800/54

(57) ABSTRACT

Methods and compositions are provided for the non-invasive diagnosis of likelihood of HIV remission after antiretroviral therapy (ART) interruption, prediction of time-to-viral-rebound in an HIV subject after ART interruption and prediction of the probability of viral rebound in an HIV subject after ART interruption. Methods for treating HIV subjects by increasing or decreasing expression of certain metabolites or glycans are also provided.

### STATEMENT REGARDING FEDERALLY SPONSORED RESEARCH OR DEVELOPMENT

This invention was made with government support under grant numbers AI143385 and CA010815 awarded by the National Institutes of Health. The government has certain rights in this invention.

### BACKGROUND OF THE INVENTION

Several therapeutic strategies are being tested in clinical trials to reduce the size of HIV reservoirs to a point where virologic control can be achieved without antiretroviral therapy (ART). The success of these strategies depends on the capacity to determine if potential interventions have made a meaningful impact on the HIV reservoir, i.e. if they have extended the likely period of ART-free remission following treatment discontinuation. Because current technologies are unable to measure the impact of interventions on the total body burden of HIV, HIV cure-focused clinical trials rely on the inclusion of an Analytical Treatment Interruption (ATI) as the only definitive approach to evaluate the effectiveness of interventions. However, this approach is costly, cumbersome, and poses some risk to both study participants and the community.

In the last few years, a small number of immunophenotypic and virologic measurements have been associated with time-to-viral-rebound. Levels of exhaustion markers on CD4+ T cells, measured pre-ART, correlated with time-to-rebound. However, these measures fail as biomarkers when assessed during ART. Levels of cell-associated HIV DNA and RNA, as well as features of plasmacytoid dendritic cells, during ART, correlate with viral rebound after ART cessation; however, the correlations are generally weak. Thus, as of now, there are no sufficiently reliable or validated biomarkers that can be leveraged to guide clinical decision making.

### SUMMARY OF THE INVENTION

In one aspect, a diagnostic kit for the non-invasive prediction of time-to-viral rebound in an HIV subject after antiretroviral therapy (ART) interruption comprises reagents that detect and measure the metabolites and glycans comprising Plasma A3G3S3 glycans, Pyruvic acid, Plasma T-antigen that binds the lectin MPA, Plasma T-antigen that binds the lectin ACA, Total fucose, L-glutamic acid, and N-acetyl-glucosamine ((GlcNAc)n).

In another aspect, a diagnostic kit for the non-invasive prediction of the probability of viral rebound in an HIV subject after antiretroviral therapy (ART) interruption comprises reagents that detect and measure the metabolites and glycans comprising IgG A2 glycans, Plasma A3G3S3 glycans, Total Fucose, Core fucose, Plasma T-antigen that bind the lectin ABA, (GlcNAc)n and L-glutamic acid.

In yet another aspect, a method for predicting time-to-viral rebound in an HIV-infected subject's sample, comprises detecting and measuring the levels of the combination of metabolites and glycans comprising Plasma A3G3S3 glycans, Pyruvic acid, Plasma T-antigen that binds the lectin MPA, Plasma T-antigen that binds the lectin ACA, Total fucose, L-glutamic acid, and (GlcNAc)n.

In still another aspect, a method for the non-invasive prediction of the probability of viral rebound in an HIV subject after antiretroviral therapy (ART) interruption comprises detecting and measuring the metabolites and glycans comprising IgG A2 glycans, Plasma A3G3S3 glycans, Total Fucose, Core fucose, Plasma T-antigen that bind the lectin ABA, (GlcNAc)n and L-glutamic acid.

In yet another aspect, a method for treating an HIV-infected subject comprises increasing the in vivo level or expression of one of more of the metabolites and glycans. In certain embodiments, these metabolites and glycans are one or more of Glychocholic acid, Taurochenodeoxycholic acid, Glycoursodeoxycholic acid, D Glucose, Ethylmalonic acid, N-Acetyl glutamic acid, Malonic acid, Ethylmalonic acid, 1, 5, Anhydro D glucitol, Kojic acid, D-ribono-1,4-lactone, Gamma aminobutyric acid GABA, Oxoglutaric acid (α-ketoglutaric acid) and L-glutamic acid.

In still another aspect, a method for treating an HIV-infected subject comprises decreasing the in vivo level or expression of one of more of certain metabolites and glycans. In certain embodiments, these metabolites and glycans are one or more of Pyruvic acid, L-Lactic acid, Taurine, Glycerol-3-phosphate, Indole-3 lactic acid, Indole 3 pyruvic acid, Imidazole lactic acid, Glycerophospho-N-palmitoyl ethanolamine, 2-oxindole, 3-Indoxyl sulphate, Nicotinamide, and Trimethylamine N-oxide.

Still other aspects and advantages of these compositions and methods are described further in the following detailed description of the preferred embodiments thereof.

### BRIEF DESCRIPTION OF THE DRAWINGS

FIG. 1A-FIG. 1D show plasma metabolites associate with time-to-viral-rebound in the Philadelphia Cohort. (FIG. 1A) Cox proportional-hazards model of metabolites that associate with longer (lower 13 metabolites on left side of vertical line) or shorter (top 12 metabolites on right side of vertical line) time-to-viral rebound during ATI. False Discovery Rate (FDR) was calculated using Benjamini-Hochberg correction. (FIG. 1B) Mantel-Cox test analysis of four selected metabolites from FIG. 1A. Low pre-ATI levels=lower than group median; High pre-ATI levels=higher than group median. (FIG. 1C) Pathway analysis of the 13 metabolites (lower 13 metabolites in table of FIG. 1A) whose pre-ATI levels associated with delayed viral rebound. Left image: multi-analysis approach combining KEGG and STRING Interaction Network. Right image: unbiased analysis using MetaboAnalyst 3.0 where the node color is based on p-value, and the node radius is based on the pathway impact value. The pathway impact is determined by normalizing the sum of matched metabolites to the sum of all metabolites in each pathway. (FIG. 1D) Pathway analysis of the 12 metabolites (top 12 metabolites from pyruvic acid to Trimethylamine N-oxide in FIG. 1A) whose pre-ATI levels associated with an accelerated viral rebound. Analysis was performed as in FIG. 1C.

FIG. 2A-FIG. 2F show L-glutamic acid and Pyruvic acid directly impact latent HIV reactivation and/or macrophage inflammation. (FIG. 2A) JLat 5A8 or 10.6 clones were stimulated with appropriate stimuli in the presence or absence of L-glutamic acid or vehicle control (cell culture suitable HCl solution). Geometric mean fluorescence intensity (MFI) of HIV-regulated GFP expression was measured by flow cytometry. Cell viability was determined by LIVE/DEAD aqua staining. (FIG. 2B) J-Lat 5A8 cells (n=3) and (FIG. 2C) J-Lat 10.6 cells (n=3), were treated with PMA/I (2 nM/500 nM), ImmunoCult Human CD3/CD28 T Cell Activator (25 μl per 10⁶ cells), or TNFα (10 ng/ml) in the presence or absence of L-glutamic acid (5 mM) or appropriate control. Bar graphs display mean±SD values, and statistical comparisons were performed using two-tailed unpaired t-tests. (FIG. 2D) THP-1 cells were differentiated into macrophage-like cells using PMA. Cells were then treated with L-glutamic acid (5 mM), Pyruvic acid (2 mM), or appropriate controls for 2 hours prior to LPS/IFNγ stimulation for 5 hours. Cell viability was determined by LIVE/DEAD aqua staining and cytokine secretion was measured in the supernatants using ELISA and MSD platform multiplex assay (FIG. 2E) L-glutamic acid significantly inhibited LPS/IFNγ-mediated secretion of pro-inflammatory cytokines such as IL-6 and TNFα but significantly increased the anti-inflammatory IL-10 release. Bar graphs display mean±SD, and statistical comparisons were performed using two-tailed unpaired t-tests. (FIG. 2F) Pyruvate significantly increased LPS/IFNγ-mediated secretion of IL-6 and TNFα. Bar graphs display mean±SD, and statistical comparisons were performed using two-tailed unpaired t-tests.

FIG. 3 shows hazard ratios of plasma glycomic and metabolic markers that associated with time-to-viral-rebound in the ACTG Cohort. Cox proportional-hazards model of glycomic and metabolic markers of time to (top panel) VL≥1000 or (bottom panel) two constitutive VL≥1000 within the ACTG Cohort. G=group (All=using data from all 74 participants and PTC=using data from only the 27 PTCs within the ACTG Cohort). False Discovery Rate (FDR) was calculated using Benjamini-Hochberg correction.

FIG. 4 shows Mantel-Cox plots of plasma glycomic and metabolic markers that associated with time-to-viral-rebound in the ACTG Cohort. Graphic representation of Mantel-Cox test illustrating six glycans and one metabolite that predicted time-to-viral-rebound in FIG. 3. Low pre-ATI levels=lower than the median; high pre-ATI levels=higher than the median.

FIG. 5A-FIG. 5F show plasma glycomic and metabolic markers of time-to-viral-rebound associate with levels of PBMC-associated HIV DNA (total, intact, and defective) and RNA in the ACTG Cohort. (FIG. 5A) Two-sided Spearman's correlation heat-map showing associations between markers associated with time-to-viral-rebound (in rows) and levels of cell-associated HIV DNA and RNA (measured by qPCR) or levels of intact, defective, and hypermutated HIV DNA (measured by near-full length sequencing) (in columns). The size and color of circles represent the strength of the correlation. Numbers inside the circles are nominal P values. (FIG. 5B, FIG. 5C) Inverse associations between pre-ATI plasma levels of total fucose and levels of pre-ATI cell-associated (FIG. 5B) HIV DNA or (FIG. 5C) HIV RNA. (FIG. 5D) Positive association between pre-ATI plasma levels of the metabolite pyruvic acid and levels of cell-associated HIV DNA. (FIG. 5E, FIG. 5F) Inverse associations between pre-ATI plasma levels of the metabolite L-glutamic acid and levels of intact (FIG. 5E) or defective (FIG. 5F) HIV DNA. All correlations were done using two-sides Spearman's rank correlation coefficient tests. For all panels, n=32, 31, 19, 19, and 19 biologically independent samples were used for correlations with total HIV DNA (by qPCR), cell-associated HIV RNA (by qPCR), intact HIV DNA (by sequencing), defective HIV DNA (by sequencing), and hypermutated HIV DNA (by sequencing), respectively.

FIG. 6A-FIG. 6J show plasma glycomic and metabolic markers that distinguish post-treatment controllers (PTCS) from non-controllers (NCs). Pre-ATI levels of three glycan structures are lower in PTCs compared to NCs: (FIG. 6A) the disialylated glycans, A2, in the IgG glycome, (FIG. 6B) the highly sialylated glycan structure (A3G3S3), and (FIG. 6C) T/Tn antigen (measured as binding to ABA lectin). Pre-ATI levels of four glycan structures were higher in PTCs compared to NCs: (FIG. 6D) total fucose (binding to AAL lectin) in plasma, (FIG. 6E-FIG. 6F) core fucose (binding to LCA lectin) in plasma, and (FIG. 6G-FIG. 6H) (GlcNAc)n (binding to STL and UDA lectins). Pre-ATI levels of two metabolites were higher in PTCs compared to NCs: (FIG. 6I) α-ketoglutaric acid and (FIG. 6J) L-Glutamic acid. All statistical comparisons were performed using a Mann-Whitney test. Truncated violin plots showing median. False Discovery Rate (FDR) was calculated using Benjamini-Hochberg (BH) correction.

FIG. 7A-FIG. 7B show a multivariable logistic model using machine learning predicts probability-of-viral-rebound by 97.5%. The machine learning algorithm, Lasso (least absolute shrinkage and selection operator) regularization, selected seven markers from FIGS. 6A-FIG. 6E, and FIG. 6J. Analysis using this model demonstrates that when these seven markers are combined, their predictive ability is better than the predictive ability of any marker individually (FIG. 22). (FIG. 7A) Receiver operator characteristic (ROC) curve showing result from a multivariable logistic regression model that combined these seven variables. (FIG. 7B) Results from the multivariable logistic model were translated into a probability-for-viral-rebound (PVR) score for each individual and then tested for the ability to accurately classify PTCs and NCs. The model was able to correctly classify 97.7 of NCs (97.7% sensitivity), 85.2% of PTCs (85.2% specificity) with overall accuracy of 92.9%. Squares represent individuals the model failed to correctly identify.

FIG. 8 shows longitudinal viral loads of PTCs and NCs from the ACTG cohort.

FIG. 9A-FIG. 9D show pathways enriched in the analysis of plasma metabolites for time-to-viral-rebound in the Philadelphia Cohort. (FIG. 9A) Glutamate metabolism pathway. (FIG. 9B) Primary bile acid biosynthesis pathway. (FIG. 9C) Pyruvate metabolism pathway. (FIG. 9D) Tryptophan metabolism pathway. Metabolites (L-Glutamic acid, N-Acetyl glutamic acid, Oxoglutaric acid (α-ketoglutaric acid), Gamma aminobutyrate, Ethylmalonic acid, Malonic acid, Glycocholic acid, Glycoursodeoxycholic acid, Taurochenodeoxycholic acid) are those in which their pre-ATI levels associated with a delayed viral rebound. Metabolites (Pyruvic acid, Lactic acid, Glycerol 3-phosphate, 2-Oxindole, 3-Indoxyl sulphate, Indole-3-pyruvic acid, Indole-3-lactic acid and Taurine) are those in which their pre-ATI levels associate with accelerated viral rebound.

FIG. 10A-FIG. 10B show L-glutamic acid inhibits latent HIV reactivation in a glutamine-free media. JLat 5A8 cells were stimulated with PMA/I (2 nM/500 nM) in the presence or absence of L-glutamic acid (4, 5, or 6 mM) or vehicle control (cell culture suitable HCl solution). (FIG. 10A) Cell viability was determined by LIVE/DEAD aqua staining. (FIG. 10B) Geometric mean fluorescence intensity (MFI) of HIV-regulated GFP expression was measured by flow cytometry. n=3 independent experiments. Bar graphs display mean±SD values, and statistical comparisons were performed using two-tailed unpaired t-tests. PMA/I=phorbol-12-myristate-13-acetate/ionomycin.

FIG. 11A-FIG. 11B show effects of L-Glutamic acid and pyruvic acid on myeloid inflammation. THP-1 cells were differentiated into macrophage-like cells using PMA. Macrophage-like cells were treated with L-Glutamic acid, Pyruvic acid, or appropriate controls for 2 hours before stimulating with LPS and IFNγ for 5 hours. (FIG. 11A) Impact of L-Glutamic acid on LPS/IFNγ mediated cytokine secretion. (FIG. 11B) Impact of Pyruvate on LPS/IFNγ mediated cytokine secretion. Mean±SD is displayed as bar charts, and statistical comparisons were performed using two-tailed unpaired t-tests.

FIG. 12 shows the structures and names of N-glycans identified in plasma by capillary electrophoresis.

FIG. 13 shows the structures and names of N-glycans identified in IgG by capillary electrophoresis.

FIG. 14A-FIG. 14F show levels of cell-associated HIV DNA and RNA distinguish PTCs from NCs and predict time-to-viral-rebound. (FIG. 14A-FIG. 14E) Pre-ATI levels of total cell-associated HIV DNA, measured by qPCR (FIG. 14A), cell-associated HIV RNA, measured by qPCR (FIG. 14B), intact HIV DNA, measured by sequencing (FIG. 14C), defective HIV DNA, measured by sequencing (FIG. 14D), and hyper-mutated HIV DNA, measured by sequencing (FIG. 14E), are lower in PTCs compared to NCs. All statistical comparisons were performed using a two-sided Mann-Whitney test. Truncated violin plots showing median (line in the middle), 25th percentile (line below the median line), and 75th percentile (line above the median line). (FIG. 14F) Cox proportional-hazards model was used to analyze the associations between cell-associated HIV DNA or RNA and longer or shorter time-to-viral rebound during ATI. Log 2 transformed data were used for analysis. Nominal two-sided P value of each independent variable in the model was used. Data are presented as hazard ratios with 95% confidence intervals. For all panels, n=32, 31, 19, 19, and 19 biologically independent samples were used for analyses of total HIV DNA (by qPCR), cell-associated HIV RNA (by qPCR), intact HIV DNA (by sequencing), defective HIV DNA (by sequencing), and hyper-mutated HIV DNA (by sequencing), respectively.

FIG. 15 is a table showing clinical and demographic data of the Philadelphia Cohort.

FIG. 16A and FIG. 16B are tables showing demographic and clinical characteristics of the ACTG cohort.

FIG. 17 is a table showing a list of metabolites whose pre-ATI levels associate with time-to-viral-rebound upon ART cessation.

FIG. 18 is a table showing pathways associated with time-to-viral-rebound in the Philadelphia Cohort.

FIG. 19 is a table showing lectins used in the 45-plex lectin microarray and their glycan-binding specificity.

FIG. 20 is a table showing plasma glycomic and metabolomic predictors of time-to-viral-rebound using samples from the ACTG Cohort after correction for potential confounders.

FIG. 21 is a table showing comparisons of C-index values between each univariate Cox model and the multivariable Cox model with Lasso selected variables predicting time to VL≥1000 copies/ml. This table discloses a set of plasma glycans and metabolites useful to predict time-to-viral-rebound. The seven biomarkers which in combination are useful to provide a signature predicting time to viral rebound are shown in bold, namely, Plasma A3G3S3 glycans, Pyruvic acid, Plasma T-antigen (MPA binding), Plasma T-antigen (ACA binding), Total fucose (AAL binding), L-glutamic acid, and (GlcNAc)n (STL binding).

FIG. 22 is a table showing comparisons of AUC C-index values estimated from 5-fold cross-validated models with Lasso selected variables.

FIG. 23 is a table showing plasma glycomic and metabolic markers that distinguished PTCs from NCs in the ACTG Cohort after correction for potential confounders.

FIG. 24 is a table showing comparisons of AUC values between each logistic model employing single marker predictors versus multivariable logistic regression with Lasso selected marker predicting PTC and NC status. This table discloses a set of plasma glycans and metabolites useful to predict probability-of-viral-rebound. The seven biomarkers which in combination are predictive of the probably of a good outcome are shown in bold print, namely IgG A2 glycans, Plasma A3G3S3 glycans, Total Fucose (AAL binding), Core fucose (LCA binding), Plasma T-antigen (ABA binding), (GlcNAc)n (STL binding) and L-glutamic acid.

FIG. 25 is a table showing relative standard deviation (RSD) values of the significant metabolites in the Philadelphia and/or ACTG cohorts.

FIG. 26A-FIG. 26C show plasma and IgG glycomic biomarkers of time-to-viral-rebound and viral setpoint in the Philadelphia cohort of Example 11. (FIG. 23A) Cox proportional hazards model of glycomic biomarkers identified using samples from the Philadelphia cohort. F, fucose; G, galactose; S, sialic acid; B, bisected GlcNac. Plasma*: data generated using lectin microarray; remaining data generated using capillary electrophoresis. (FIG. 23B) Mantel-Cox model illustrating two glycan structures from FIG. 23A. (FIG. 23C) Pre-ATI plasma glycomic signatures associated with a higher or a lower viral setpoints post-ATI in the Philadelphia cohort. Pearson correlation coefficient. ATI, analytic treatment interruption.

FIG. 27 shows initial validation of the glycomic signature in the Johannesburg cohort. Mann-Whitney test (for non-normally distributed data sets) or unpaired t-test (for normally distributed data sets) of Example 11. VL, viral load. Mean±SEM.

### DETAILED DESCRIPTION

The methods and compositions described herein meet the need in the art for biomarkers and diagnostic methods and compositions or kits that can accurately predict time-to-viral-rebound after treatment interruption and can be leveraged to guide clinical decision making. Such predictive biomarkers are useful to improve the safety of ATIs and accelerate the development of an HIV cure by providing a means for selecting only the most promising therapies for testing by ATIs. These biomarkers also provide important insights into the molecular and biochemical pathways and mechanisms involved in post-ART control of HIV and those underlying the clinically-relevant and desirable phenotype PTC. In other methods and compositions described herein, the inventors identify certain glycans and metabolites, which when increased or decreased in vivo in a subject with HIV can provide a phenotype that increases the subject's response to other ART or regimens.

Technical and scientific terms used herein have the same meaning as commonly understood by one of ordinary skill in the art to which this invention belongs and by reference to published texts, which provide one skilled in the art with a general guide to many of the terms used in the present application. The definitions contained in this specification are provided for clarity in describing the components and compositions herein and are not intended to limit the claimed invention.

The term “anti-retroviral therapy” or “ART” refers to treatment of individuals infected with human immunodeficiency virus (HIV) using anti-HIV drugs. The standard treatment consists of a combination of at least three drugs (often called “highly active antiretroviral therapy” or HAART) that suppress HIV replication. Antiretroviral medicines that are often used to treat HIV include: Nucleoside/nucleotide reverse transcriptase inhibitors, also called nucleoside analogs, such as abacavir, emtricitabine, and tenofovir. These medicines are often combined for best results. Nonnucleoside reverse transcriptase inhibitors (NNRTIs), such as efavirenz, etravirine, and nevirapine. Protease inhibitors (PIs), such as atazanavir, darunavir, and ritonavir. Entry inhibitors, such as enfuvirtide and maraviroc. Integrase inhibitors, such as dolutegravir and raltegravir. In one embodiment, ART is a combination of drugs efavirenz, tenofovir, and emtricitabine. Other combinations, without limitation, include: Dolutegravir, abacavir and lamivudine, Dolutegravir, tenofovir and emtricitabine, elvitegravir, cobicistat and tenofovir, and emtricitabine, raltegravir, tenofovir and emtricitabine, or ritonavir-boosted darunavir, tenofovir and emtricitabine.

The term Analytical Treatment Interruption (ATI) refers to an ART regimen in which treatment is interrupted to allow for further evaluation of the efficacy of treatment and suitability of the subject for different forms of ART or curative therapies.

The term “glycan” as used herein refers to a complex oligosaccharide composed of 10-15 monosaccharide residues. One or more glycan(s) can be covalently attached to a protein to form a glycoprotein(s), or to a lipid(s) to form a glycolipid(s). Most human proteins are modified by covalent attachment of glycans. Most glycans attached to proteins can be classified as N-glycans, attached through nitrogen of asparagine, or O-glycans, attached through oxygen of mainly serine or threonine. Glycans of interest can include, without limitation, one or more of monosialylated structures, di-sialylated structures, trisialylated structures, tetrasialylated structures, agalactosylated structures, monogalactosylated structures, di-galactosylated structures, trigalactosylated structures, tetragalactosylated structures, low branched (monoantennary and diantennary) structures, high branched (triantennary and tetraantennary), structures with bisecting GlcNAc, antennary fucosylated structures, and core fucosylated structures.

The term “glycome” as used herein refers to the set of all glycans in an organism/tissue/cell, or even of a single glycoprotein. In one embodiment, the glycome is the set of all glycans in the IgG of a human subject. In another embodiment, the glycome is the set of all glycans in the plasma of a human subject. In another embodiment, the glycome is the set of all glycans on the subject's cell-surface, either from all cells or from a selected cell type. In another embodiment, the glycome is the set of all glycans in the subject's tissues, either from all tissues or from a selected tissue type. In another embodiment, the glycome is the subject's total exosome-bound glycome.

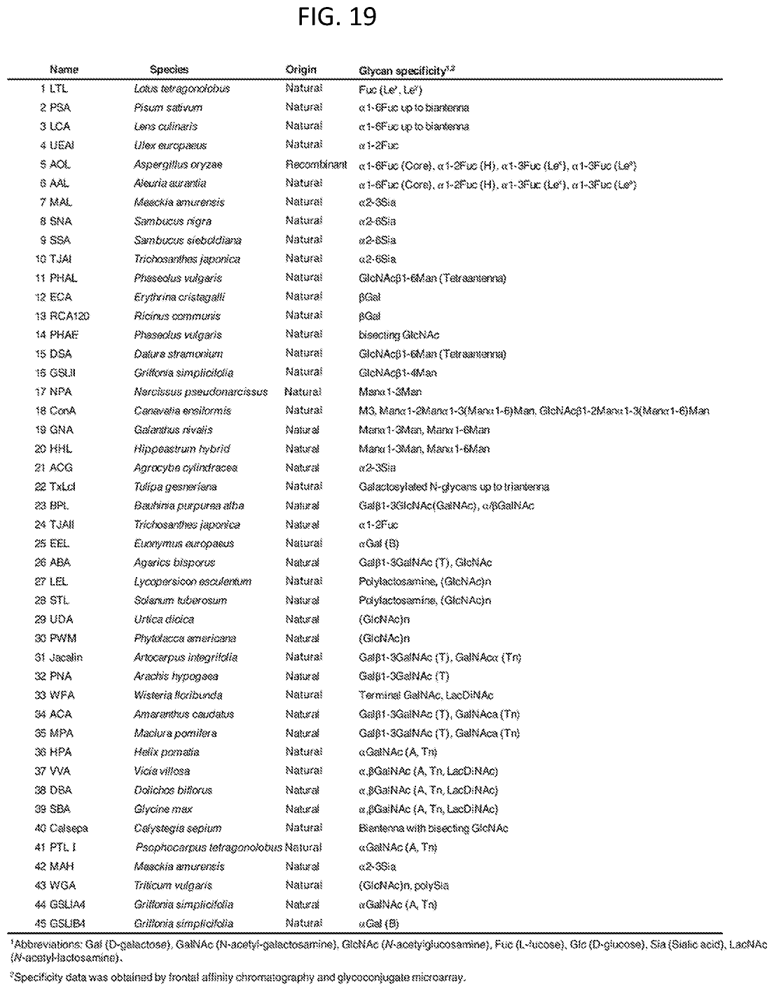

The term “plasma metabolites” refers to biologically active molecules that function to regulate critical immunological responses, including inflammatory responses. The term “lectin” refers to a protein with a functional carbohydrate recognition domain which binds specific glycan structures, regarding both monomer composition and spatial arrangement. The lectins referred to throughout the specification by abbreviations, e.g., ABA binding, STL binding, etc., are specifically identified in the table of FIG. 19.

“Patient” or “subject” or “individual” as used herein mean a mammalian animal, including a human, a veterinary or farm animal, a domestic animal or pet, and animals normally used for clinical research. In one embodiment, the subject of these methods and compositions is a human. In one embodiment, the subject of these methods is a human person with HIV. The subject in one embodiment is an HIV+ subject who has received or currently is receiving antiretroviral therapy (ART). In another embodiment, the subject is a person with HIV who is experiencing a treatment interruption. In another embodiment, the subject is a person with HIV who is being screened for a clinical trial. In another embodiment, the subject is a person with HIV who is a PTC. In another embodiment, the subject is a person with HIV who may receive a change in ART regimen or protocol. In still another embodiment, the subject is a person with HIV who is anticipating undergoing a curative therapy or combination or therapies for HIV.

“Post-treatment controllers” or “PTCs” refer to a rare population of individuals that demonstrate sustained virologic suppression for several months to years after ART cessation. The mechanisms underlying viral control in these individuals are not completely understood. PTC represent a clinically relevant model for viral control post-ART. In contrast, the majority of HIV-infected individuals experience rapid viral rebound after ART interruption. The existence of individuals with the PTC phenotype was employed in the data discussed herein to define a set of biomarkers that can predict the probability-of-viral-rebound after potentially successful intervention (i.e., the likelihood to achieve a PTC phenotype after ART cessation).

“Sample” as used herein means any biological fluid or suspension or tissue from a subject, that contains metabolites and/or glycomic biomarkers as identified herein. The most suitable samples for use in the methods and with the diagnostic compositions or reagents described herein are samples or suspensions which require minimal invasion for testing, e.g., total plasma or isolated immunoglobulin G (IgG). Other samples which can be manipulated for measurement of such biomarkers include blood samples, including whole blood, peripheral blood, or serum, as well as cerebrospinal fluid, serous fluid, saliva or urine, vaginal or cervical secretions, and ascites fluids or peritoneal fluid or tissues containing HIV reservoirs. In another embodiment, a suitable sample for use in the methods described herein includes peripheral blood, more specifically peripheral blood mononuclear cells. Any sample containing the biomarkers may be similarly evaluated by the methods described herein. In one embodiment, such samples may further be treated to isolate the indicated glycoproteins modified by selected glycans. In another embodiment, the samples are concentrated by conventional means.

Control, control level, control signature or control profile as used herein refers to the source of the reference glycomic or metabolic biomarker against which the tested subject's biomarker or combination of biomarkers are analyzed, i.e., the levels of one or more selected glycans, metabolites or total plasma, IgG, circulating, or HIV reservoir glycans or metabolites in a specified subject or in an average population of multiple subjects having a common condition or stage of disease. In one embodiment, the reference controls are obtained from biological samples selected from a reference healthy non-HIV-infected human subject or average population of such subjects. In another embodiment, the reference biomarkers utilized is a signature or group of biomarkers derived from biological samples of a reference human subject or population of human subjects who are post-ART and demonstrate no HIV comorbidities and/or no HIV reservoirs. In one embodiment the reference population comprises Immune Responders (IR). In another embodiment the reference population comprises Immune Non-Responders (INR). In another embodiment, the reference population are PTCs. In certain embodiments, the reference glycomic signature or profile utilized is a profile derived from a reference human subject, or an average of multiple subjects, with specific early stage HIV symptoms or co-morbidities. In another embodiment, the reference signature or profile is a standard or profile derived from a reference human subject, or an average of multiple subjects, with late stage symptoms or co-morbidities. In another embodiment, the reference is a profile derived from the biological samples of the same human subject at a prior time, e.g., before or after ART or ATI, or before or after other HIV treatment or curative treatment. The control or reference standard, in various embodiments, is a mean, an average, a numerical mean or range of numerical means, a numerical pattern, a graphical pattern or a nucleic acid or gene expression profile derived from a control subject or a control population.

By “diagnosis” or “evaluation” as used herein is meant the diagnosis or prediction of time to viral rebound of HIV infection after treatment interruption. In one embodiment, a diagnosis of delay of viral rebound is based on the presence in the subject's plasma of certain key metabolites or glycans, or levels of same, present in plasma that indicate that the patient is in good condition to increase response to other ART or curative treatments. In another embodiment, a diagnosis of early viral rebound is based on the presence in the subject's plasma of certain key metabolites or glycans, or levels of same, present in plasma that indicate that the patient is in need of additional ART before other curative treatments are useful. These diagnoses are useful to improve the safety of ATI, accelerate the development or curative strategies, and provide biological clues to the molecular mechanistic underpinnings of HIV persistence.

As used herein, the term “treatment” refers to any method used to alleviate, delay onset, reduce severity or incidence, or yield prophylaxis of one or more symptoms or aspects of HIV. For the purposes of the present invention, treatment can be administered before, during, and/or after the onset of symptom. In certain embodiments, treatment occurs after the HIV+ subject has received ART. In some embodiments, the term “treating” includes abrogating, substantially inhibiting, slowing, or reversing the progression of a condition, substantially ameliorating clinical or aesthetical symptoms of a condition, or substantially preventing the appearance of clinical or aesthetical symptoms of HIV, or decreasing the severity and/or frequency one or more symptoms resulting from the disease. More specifically treatment as described herein includes manipulating the level of the selected glycan or metabolite in vivo to reduce HIV+ persistence, decrease the HIV+ reservoir, and/or reduce the severity, delay the onset, or prevent the development of a HIV comorbidity. In one embodiment, where the metabolite or glycan is L-glutamic acid, treatment involves administering L-glutamic acid-containing compositions or therapeutic agents that operate to increase the levels of L-glutamic acid in the subject. In another embodiment, where the metabolite or glycan is pyruvic acid, treatment involves administering compositions or therapeutic agents that operate to decrease the levels of pyruvic acid in the subject.

By “diagnostic reagent” as used herein is meant any compositions that can be used to detect or measure the levels of one or multiple metabolites or glycans in the subject's body. In one embodiment, the reagent is a lectin that detects the sugar on the glycan. In another embodiment, the reagent is an antibody or antibody fragment that detects the glycan/metabolite protein. Such reagents can be labeled with detectable labels, such as fluorescent labels. Still other reagents are those which are normally used in the protocols of mass spectrometry, capillary electrophoresis, lectin microarray, ELISA using a lectin rather than an antibody, lectin blotting and lectin hybridization.

By “therapeutic agent” as used herein means any compositions that can be used to manipulate or modify the levels of one or multiple metabolites or glycans in the subject's body to place the subject in condition for further HIV treatments or curative therapies. In one embodiment, the therapeutic agent is a selected glycan or metabolite in or associated with a suitable pharmaceutical carrier or excipient, such as a nanoparticle coated with one or more glycans or metabolites. As discussed herein, and in the examples below, an L-glutamic acid coated nanoparticle may be administered to a subject demonstrating fast viral rebound after ATI. In another embodiment, the therapeutic reagent is a conjugate formed of a targeting moiety and the glycan/metabolite. In still another embodiment, the therapeutic reagent can include a compound or precursor in the selected glycan/metabolite biosynthetic pathway or derivative thereof, e.g., for example a precursor in the L-glutamic acid pathway. In still another embodiment, the therapeutic reagent is an inhibitor of the glycan or metabolite or precursor in the glycan or metabolite biosynthetic pathway or derivative thereof or a glycosylation inhibitor or deglycosylation enzyme, which can reduce the over-production of the selected glycan or metabolite. In one embodiment, such reduction is temporary to enable the subject to receive combination therapies for treatment or cure of HIV. Still other therapeutic reagents can include compounds or chemical moieties that can manipulate expression of the metabolites and glycans identified herein. Any of the active therapeutic reagents can be associated with known carriers or targeting compositions, such as taught in the prior art.

The terms “a” or “an” refers to one or more. For example, “a biomarker” is understood to represent one or more such biomarkers. As such, the terms “a” (or “an”), “one or more,” and “at least one” are used interchangeably herein.

As used herein, the term “about” means a variability of plus or minus 10% from the reference given, unless otherwise specified.

The words “comprise”, “comprises”, and “comprising” are to be interpreted inclusively rather than exclusively, i.e., to include other unspecified components or process steps. The words “consist”, “consisting”, and its variants, are to be interpreted exclusively, rather than inclusively, i.e., to exclude components or steps not specifically recited.

In performance of the methods described herein and in assembly of the components of the compositions, all disclosures, methods and materials used in the Examples below may be employed.

### Compositions or Kits

In one embodiment, a composition is a kit is provided that contains reagents that detect and/or measure levels of certain metabolites and glycans which in combination enable a non-invasive prediction of time-to-viral rebound in an HIV subject after antiretroviral therapy (ART) interruption. Such a kit can contain reagents that detect or measure Plasma A3G3S3 glycans. Such a kit can contain reagents that detect or measure Pyruvic acid. Such a kit can contain reagents that detect or measure Plasma T-antigen that binds lectin MPA. Such a kit can contain reagents that detect or measure Plasma T-antigen that binds lectin ACA. Such a kit can contain reagents that detect or measure Total fucose. Such a kit can contain reagents that detect or measure L-glutamic acid. Such a kit can contain reagents that detect or measure (GlcNAc)n that binds lectin STL. In still another embodiment, a kit contains reagents that detect and/or measure two, three, four, five, six or all seven of these metabolites and glycans. In one embodiment, the kit contains at least one reagent capable of detecting or measuring a single of these metabolites or glycans. In another embodiment, the kit contains at least two, three, four, five, six or all seven reagents that each detect or measure a single metabolite or glycan. In one particular embodiment, the kit contains seven reagents, which are sufficient to measure all seven of the metabolites or glycans identified above. Still other kits can identify additional metabolites or glycans, as identified throughout this specification and its figures.

In another embodiment, a composition is a kit useful for the non-invasive prediction of the probability of viral rebound in an HIV subject after antiretroviral therapy (ART) interruption. Such a kit comprises reagents that detect and IgG A2 glycans. Such a kit can contain reagents that detect or measure Plasma A3G3S3 glycans. Such a kit can contain reagents that detect or measure Total Fucose. Such a kit can contain reagents that detect or measure Core fucose that binds lectin LCA. Such a kit can contain reagents that detect or measure Plasma T-antigen that binds lectin ABA. Such a kit can contain reagents that detect or measure (GlcNAc)n that bind STL. Such a kit can contain reagents that detect or measure L-glutamic acid. In still another embodiment, a kit contains reagents that detect and/or measure two, three, four, five, six or all seven of these metabolites and glycans. In one embodiment, the kit contains at least one reagent capable of detecting or measuring a single of these metabolites or glycans. In another embodiment, the kit contains at least two, three, four, five, six or all seven reagents that each detect or measure a single metabolite or glycan. In one particular embodiment, the kit contains seven reagents, which are sufficient to measure all seven of the metabolites or glycans identified above. Still other kits can identify additional metabolites or glycans, as identified throughout this specification and its figures.

As described above, in certain embodiments, each reagent detects and measures a single of said metabolites and glycans. In one embodiment, the reagent is a lectin, optionally associated with a detectable label. In another embodiment, the reagent is an antibody, optionally associated with a detectable label. Still other known probes, small molecules, labels and ligands known to identify the glycans and metabolites can be used as reagents. A kit can also containing the relevant reagents in multiples, if desired; optional detectable labels for same, immobilization substrates, optional substrates for enzymatic labels, as well as other laboratory items. Exemplary substrates include a microarray, chip, microfluidics card, or chamber.

In one embodiment, the composition or kit can further comprise components necessary to perform a protein binding assay. In another embodiment, the kit can contain components necessary to perform a lectin micro array, a lectin ELISA, lectin blotting or lectin hybridization as known in the art. In still another embodiment, the kit can contain components necessary to perform capillary electrophoresis or mass spectrometry on the samples.

### Diagnostic Methods

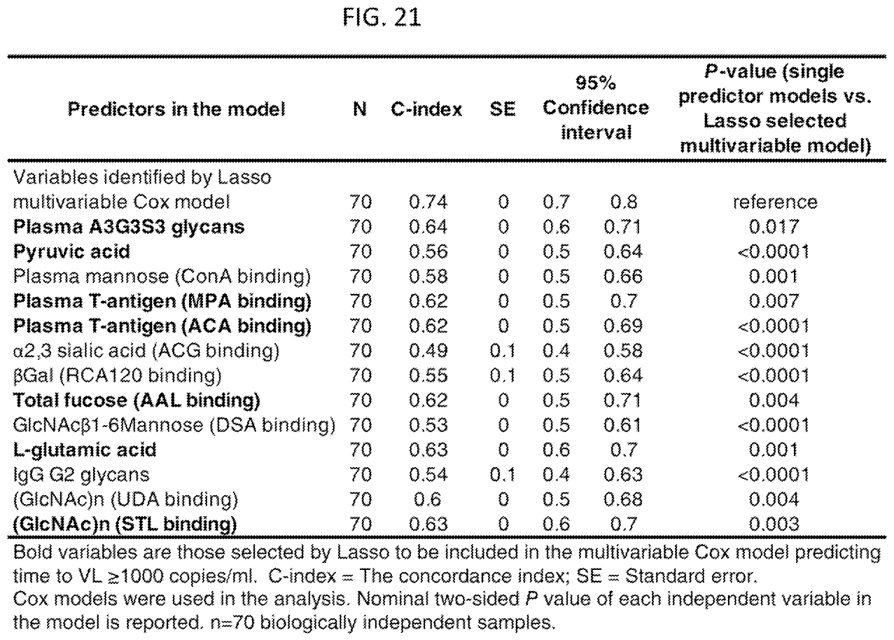

The methods can utilize the kits and reagents as described herein and in the examples for a number of different purposes. One method permits the prediction of time-to-viral rebound in an HIV-infected subject's sample by detecting and measuring the levels of a combination of metabolites and glycans. In one embodiment, the metabolites and glycans include the seven biomarkers identified in bold in FIG. 21. These biomarkers include Plasma A3G3S3 glycans, Pyruvic acid, Plasma T-antigen (MPA binding), Plasma T-antigen (ACA binding), Total fucose (AAL binding), L-glutamic acid, and (GlcNAc)n (STL binding). As demonstrated in the examples below, the method utilizing these seven metabolites/glycans can predicts time-to-viral rebound by 74-76%. Still other combinations of the biomarkers disclosed in the examples and Figures herein may be combined with one or more of these seven specifically identified biomarkers.

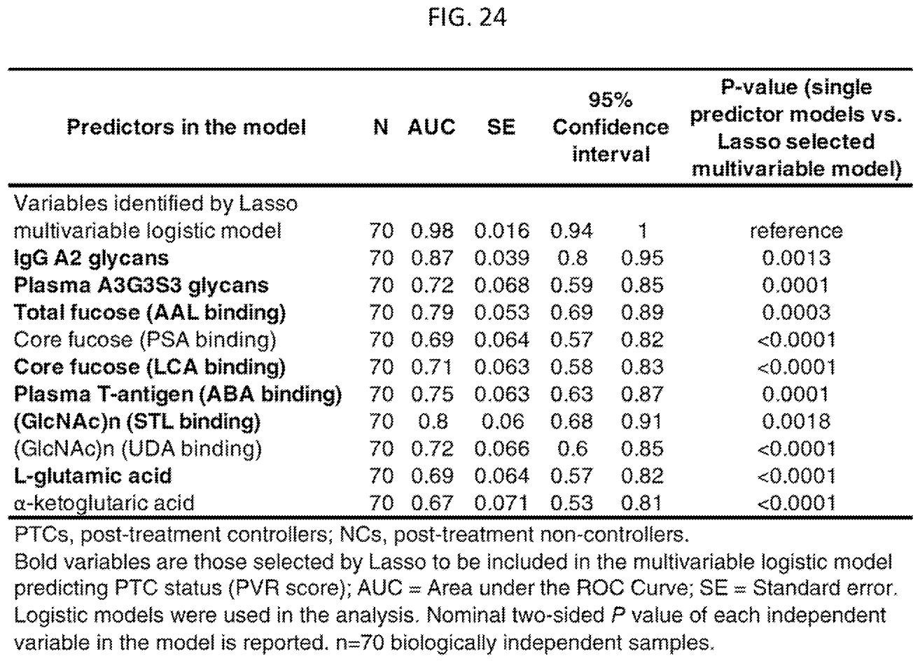

In yet another aspect, a method for the non-invasive prediction of the probability of viral rebound in an HIV subject after antiretroviral therapy (ART) interruption involves detecting and measuring another set of metabolites and glycans, as described in the examples and Figures herein. In one embodiment, this method utilizes the seven biomarkers IgG A2 glycans, Plasma A3G3S3 glycans, Total Fucose, Core fucose, Plasma T-antigen, (GlcNAc)n, and L-glutamic acid, as identified in bolded print in FIG. 24 below. As described in the examples below, this method predicts the probability of viral rebound with a sensitivity of 97.5%.

These methods are considered non-invasive because they can be performed on a subject's plasma or other biological sample. In one embodiment, these methods employ a kit as described herein. In one embodiment, either of the methods described above can employ the steps of a protein binding assay to identify and measure the biomarkers. In certain examples, the methods involve a lectin micro array, a lectin ELISA, lectin blotting or lectin hybridization. In other examples, the methods are performed using capillary electrophoresis. Still other methods of identifying and measuring the biomarkers involves the use of mass spectrometry.

Treatment of the selected subject samples to provide measurable and identifiable glycans can use techniques as described in the examples or references below. See also, US Patent Publication No. 2016/0103137, published Apr. 13, 2016, and International Patent Publication No. WO2019/140147, published Jul. 18, 2019, incorporated by reference herein. for additional method steps for preparing and measuring glycans from a plasma or IgG sample. According to the methods the glycoprotein(s) in the sample are isolated and purified. Isolation and purification methods are known in the art, for example, SDS PAGE, size exclusion chromatography, affinity resin or beads, filtration/isolation columns, various centrifugation methods to separate fractions, and the like. In one example, glycans can be separated from the pooled glycoproteins from the sample or sample component for measurement by use of treatment or digestion with an appropriate glycosidase. Individual glycans can be further segregated by use of ligands (labeled or immobilized on a solid support), such as antibodies, lectins; and then subjected to affinity purification and high-throughput analysis by HPLC. The glycan level in the test sample is determined by one or more of the following techniques, i.e., which include high-performance liquid chromatography (HPLC; e.g., normal phase or weak anion exchange HPLC), capillary electrophoresis (CE), gel electrophoresis (e.g., one or two dimensional gel electrophoresis), mass spectrometry (MS), isoelectric focusing (IEF), lectin-based microarray chromatography and/or an immunoassay (e.g., immuno-PCR, ELISA, lectin ELISA, Western blot, or lectin immunoassay) on the sample or a component thereof (e.g., plasma, IgG, serum, PBMCs or tissue lysate, a pool of isolated glycans, an isolated glycoprotein, etc.). See, e.g., US 2006/80269974; US 2006/0270048, and US 2006/0269979. In one embodiment, methods for generating a glycomic signature, and/or measuring a selected single glycan structure or multiple glycan structures within a sample can use ultra-performance liquid chromatography (UPLC), as described in the examples and references below. In other embodiments, electrospray ionization—time of flight (ESI-TOF) MS coupled with reversed-phase (RP) HPLC or size-exclusion chromatography (SEC) is used. Still other techniques include matrix assisted laser desorption ionization (MALDI) MS.

As described herein, glycomics and metabolomics heave been utilized to identify highly robust, host-specific plasma biomarkers that can predict the duration and probability of viral remission after treatment interruption. Plasma glycoproteins (including antibodies; immunoglobulin G (IgGs)) and plasma metabolites enter the circulation from tissues through active secretion or leakage. Therefore, their levels and chemical characteristics can reflect the overall status of multiple organs, making them excellent candidates for biomarkers. Indeed, glycomic features in total plasma and on IgG have been identified as biomarkers for inflammatory bowel disease, systemic lupus erythematosus, cancer, and diabetes. In addition, glycans on circulating glycoproteins have functional significance, as they play essential roles in mediating immunological functions, including antibody-dependent cell-mediated cytotoxicity (ADCC) and pro- and anti-inflammatory activities. Similarly, plasma metabolites have been investigated as diagnostic and prognostic biomarkers in several diseases such as heart disease, hepatitis, autism, Alzheimer's disease, cancer, and depression.

In a recent study (Giron et al. AIDS 34, 681-686 (2020), which is incorporated by reference herein), several plasma glycomic structures whose pre-ATI levels associate with delayed viral rebound after ART-discontinuation were identified. These were the digalactosylated glycans on bulk IgG, called G2, as well as fucose (total and core) and N-Acetylglucosamine (GlcNac) on total plasma glycoproteins. However, that study was a small pilot and did not explicitly address the potentially confounding effects of age, gender, ethnicity, duration-on-ART, time of ART initiation (treatment at early vs. chronic stage of infection), or pre-ATI CD4 count.

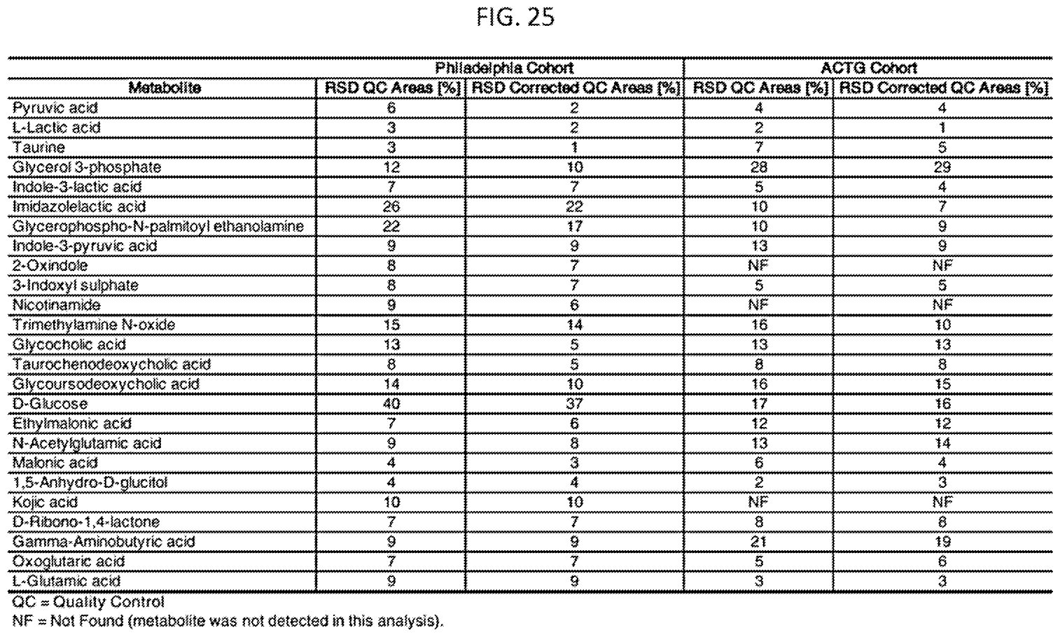

In the examples detailed below, metabolomic analysis on one of the two cohorts used in the pilot was applied. This was a cohort of 24 HIV-infected, ART-suppressed individuals who had participated in an open-ended ATI study without concurrent immunomodulatory agents. The metabolomic analysis identified several metabolites whose pre-ATI levels associate with time-to-viral-rebound. These metabolites belong to metabolic pathways known to impact inflammatory responses. The direct functional impact of some of these metabolites on latent HIV reactivation and/or macrophage inflammation in vitro was confirmed. Both the plasma glycome and metabolome of a large cohort of 74 HIV-infected, ART-suppressed individuals who underwent ATI during several AIDS Clinical Trials Group (ACTG) clinical trials were profiled. This cohort contains 27 PTCs and 47 non-controllers (NCs). Using this cohort, the utility of a set of plasma glycans and metabolites to predict time-to-viral-rebound and probability-of-viral-rebound even after adjusting for several potential demographic and clinical confounders was confirmed. Finally, using machine learning models, this set of biomarkers was combined into two multivariate models: a model that predicts time-to-viral-rebound by 74-76% (FIG. 19); and a model that predicts probability-of-viral-rebound (PVR score) by 97.5% (FIG. 25). Together, the findings fill a major gap in HIV cure research by identifying plasma non-invasive biomarkers, with potential functional significance, that predict duration and probability of viral remission after treatment interruption.

The studies described identified and validated pre-ATI plasma glycomic and metabolomic biomarkers of both duration and probability of viral remission after treatment interruption. A significant overlap between plasma markers that predicted time-to-viral rebound and markers that predicted probability of viral rebound (i.e., predicted the PTC phenotype in comparison to the NC phenotype) was observed. Specifically, pre-ATI plasma levels of the anti-inflammatory L-glutamic acid, N-Acetylglucosamine (GlcNac), and fucose were associated with both delayed rebound and higher likelihood to achieve viral remission. Whereas pre-ATI plasma levels of the highly-sialylated A3G3S3 and GalNAc-containing glycans (T/Tn-antigens) were associated with both accelerated rebound and lower likelihood of achieving viral remission. Notable differences included the digalactosylated G2 glycan on IgG glycome whose pre-ATI levels associated with longer time-to-viral-rebound but not probability of viral rebound; and the di-sialylated IgG glycan, A2, whose pre-ATI levels associated with higher probability of viral rebound but not with time-to-viral rebound.

It is not surprising that a single marker cannot highly predict these complicated virological milestones (time to and probability of viral rebound). Therefore, machine learning algorithms were applied to select the smallest number of variables that, when combined, maximizes the predictive utility of the described signatures. The variables selected by these algorithms, when used in multivariate models, were able to predict time-to-viral rebound by 74-76% (a moderate predictive value) and probability of viral rebound with a sensitivity of 97.5% (an outstanding predictive value). The utility of these multivariable models to be used in HIV cure-directed clinical trials warrants further investigation. Upon validation, these models could have a profound impact on the HIV cure field by mitigating the risk of ATI during HIV cure-focused clinical trials and provide means for selecting only the most promising therapies and most likely individuals to achieve viral remission to be tested by ATIs.

Beyond their utility as biomarkers, these metabolic and glycomic signatures of viral rebound represent an opportunity to better understand the host milieu during viral rebound. The likelihood of viral rebound and viral remission after ART cessation is likely a function of both the size of the inducible replication-competent HIV reservoir and the host environment that influences inflammatory and immunological responses. The on-going efforts by many groups to understand the quantitative and qualitative nature of the HIV reservoir are critical to understand the virological basis of viral rebound.

### Therapeutic Methods

In yet a further aspect, a method for treating an HIV-infected subject comprises increasing the in vivo level or expression of one of more of certain metabolites and glycans. In one embodiment, the method involves administering a composition to increase the level or expression in vivo of Glychocholic acid or another metabolite in its pathway. In one embodiment, the method involves administering a composition to increase the level or expression in vivo of Taurochenodeoxycholic acid or another metabolite in its pathway. In one embodiment, the method involves administering a composition to increase the level or expression in vivo of Glycoursodeoxycholic acid or another metabolite in its pathway. In one embodiment, the method involves administering a composition to increase the level or expression in vivo of D-Glucose or another metabolite in its pathway. In one embodiment, the method involves administering a composition to increase the level or expression in vivo of Ethylmalonic acid or another metabolite in its pathway. In one embodiment, the method involves administering a composition to increase the level or expression in vivo of N-Acetyl glutamic acid or another metabolite in its pathway. In one embodiment, the method involves administering a composition to increase the level or expression in vivo of Malonic acid or another metabolite in its pathway. In one embodiment, the method involves administering a composition to increase the level or expression in vivo of 1, 5, Anhydro D glucitol or another metabolite in its pathway. In one embodiment, the method involves administering a composition to increase the level or expression in vivo of Kojic acid or another metabolite in its pathway. In one embodiment, the method involves administering a composition to increase the level or expression in vivo of D-ribono-1,4-lactone or another metabolite in its pathway. In one embodiment, the method involves administering a composition to increase the level or expression in vivo of Gamma aminobutyric acid GABA or another metabolite in its pathway. In one embodiment, the method involves administering a composition to increase the level or expression in vivo of Oxoglutaric acid (α-ketoglutaric acid) or another metabolite in its pathway. In one embodiment, the method involves administering a composition to increase the level or expression in vivo of L-glutamic acid or another metabolite in its pathway.

In yet a further aspect, a method for treating an HIV-infected subject comprises decreasing or inhibiting the in vivo level or expression of one of more of certain metabolites and glycans. In one embodiment, the method involves administering a composition to decrease the level or expression in vivo of Pyruvic acid or another metabolite in its pathway. In one embodiment, the method involves administering a composition to decrease the level or expression in vivo of L-Lactic acid or another metabolite in its pathway. In one embodiment, the method involves administering a composition to decrease the level or expression in vivo of Taurine or another metabolite in its pathway. In one embodiment, the method involves administering a composition to decrease the level or expression in vivo of Glycerol-3-phosphate or another metabolite in its pathway. In one embodiment, the method involves administering a composition to decrease the level or expression in vivo of Indole-3 lactic acid or another metabolite in its pathway. In one embodiment, the method involves administering a composition to decrease the level or expression in vivo of Indole 3 pyruvic acid or another metabolite in its pathway. In one embodiment, the method involves administering a composition to decrease the level or expression in vivo of Imidazole lactic acid or another metabolite in its pathway. In one embodiment, the method involves administering a composition to decrease the level or expression in vivo of Glycerophospho-N-palmitoyl ethanolamine or another metabolite in its pathway. In one embodiment, the method involves administering a composition to decrease the level or expression in vivo of 2-oxindole or another metabolite in its pathway. In one embodiment, the method involves administering a composition to decrease the level or expression in vivo of 3-Indoxyl sulphate or another metabolite in its pathway. In one embodiment, the method involves administering a composition to decrease the level or expression in vivo of Nicotinamide or another metabolite in its pathway. In one embodiment, the method involves administering a composition to decrease the level or expression in vivo of Trimethylamine N-oxide or another metabolite in its pathway.

In certain embodiments of the treatment methods, the manipulation (increase or decrease) of the amount of the selected biomarker metabolite or glycan in vivo occurs during ART treatment. In certain embodiments of the treatment methods, the manipulation (increase or decrease) of the amount of the selected biomarker in vivo occurs subsequent to treatment of the subject with ART. In still other embodiments the methods are directed to occur between ART treatments and/or with other HIV treatments and curative therapies.

In performing the treatment methods, many therapeutic agents can be administered to the subject to modify (increase or decrease) the selected glycan or metabolite. Typical therapeutic agents useful to accomplish the increase of a selected glycan (e.g., L-glutamic acid) involve the in vivo administration of the selected glycan/metabolite in a suitable pharmaceutical carrier or excipient. In one embodiment, the agent is a carrier or nanoparticle coated with the glycan/metabolite. One such carrier is a poly (lactic-co-glycolic acid) (PLGA) nanoparticle. Still other known nanoparticles can be used for the same purpose.

The methods of treatment may also employ as a therapeutic agent a conjugate formed of a targeting moiety and the selected glycan/metabolite(s) to deliver the selected glycan/metabolite to only particular locations in the subject's body. Still additional mechanisms to increase a glycan/metabolite that is desired to be increased is employing a compound or precursor in the glycan/metabolic biosynthetic pathway or derivative thereof.

In still another treatment method in which the manipulation involves decreasing a selected glycan/metabolite involves use of an inhibitor of the glycan, metabolite or its precursor in the appropriate biosynthetic pathway or derivative thereof.

Other therapeutic agents are those that are useful in manipulating expression of the indicated glycan or metabolite, such as antibodies conjugated to glycan-manipulating molecules may be used. Also, other methods to temporarily increase or decrease or correct levels of the selected glycan or metabolite may involve genetic manipulation of the host's nucleic acids, such as by techniques such as CRISPR methods, recombinant viruses that either increase or reduce expression of a target, etc.

Additional methods of treatment are indirect and involve treating cells, e.g., PBMCs, from the subject ex vivo with glycan/metabolite coated nanoparticles to correct a deficiency in the glycan/metabolite level. Known small molecule inhibitors may also be used and selected depending upon the target metabolite/glycan.

The methods described herein may also be combined with other treatments and therapies for HIV infection.

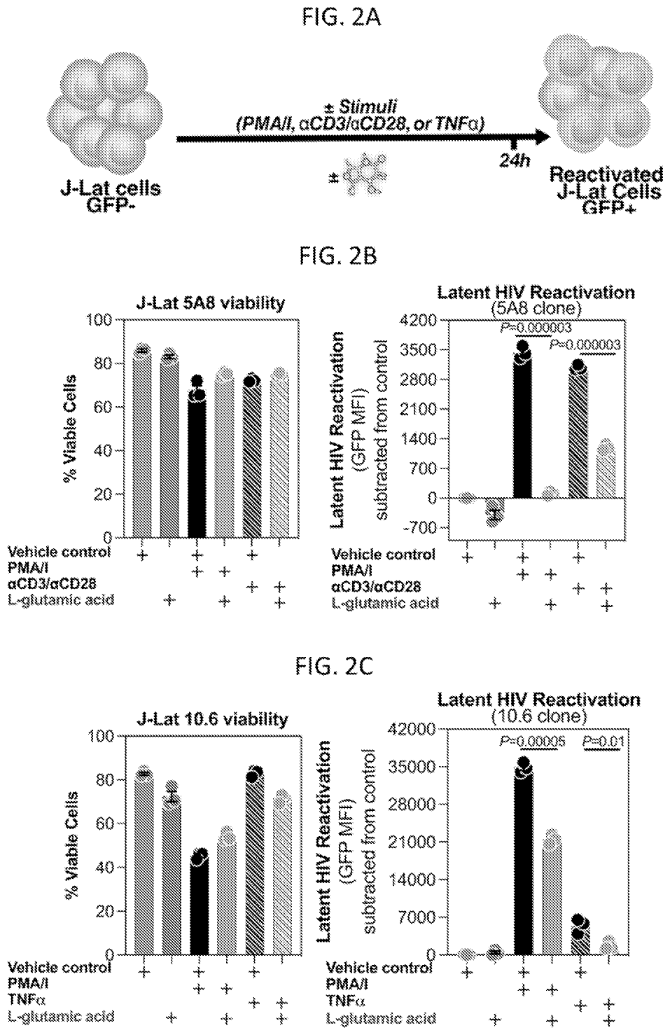

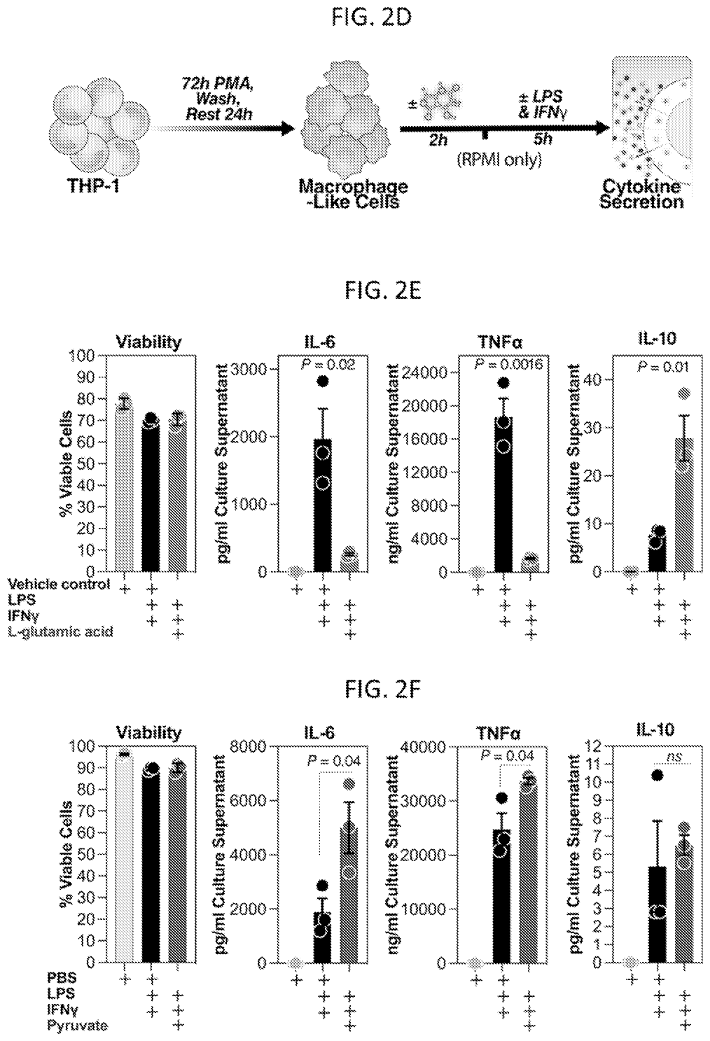

These methods are useful as host determinants of inflammatory and immunological states that also impact post-treatment control of HIV are further identified. Functional analyses on two of these biomarkers (L-glutamic acid and pyruvic acid in FIG. 2A-FIG. 2F) suggest that the signatures have a potential functional significance for HIV post-treatment control. These markers may directly impact latent HIV reactivation or may indirectly condition the host environment with differential levels of inflammation that might impact viral reactivation, cellular processes, and immunological functions during ATI.

The data obtained from two independent cohorts suggest that the bioactive plasma metabolites might not only predict duration and probability of viral remission, but also actively contribute to it. The in vivo data showed that the pre-ATI levels of L-glutamic acid predict a delayed viral rebound and a higher probability of viral remission. Indeed, the in vitro validation experiments showed that L-glutamic acid can directly suppress HIV reactivation and suppress LPS and IFNγ-mediated inflammation of myeloid cells. It has been argued that glutamate, through its conversion to α-ketoglutarate, fuels the TCA cycle/oxidative phosphorylation, which is typically regarded to be an anti-inflammatory metabolic signature. This is in agreement with the in vivo and in vitro data on L-glutamic acid.

In contrast to L-glutamic acid, in vivo data showed that elevated pre-ATI levels of the pro-inflammatory pyruvic acid associate with an accelerated viral rebound. A significant positive correlation between pre-ATI levels of plasma pyruvic acid and total CD4+ T cell HIV DNA, a marker for reservoir size, was also observed. The in vitro data confirmed these in vivo observations and showed that pyruvic acid can induce a strong pro-inflammatory phenotype in myeloid cells upon stimulation. Aerobic glycolysis, where pyruvate is converted into lactate, is enhanced in metabolically demanding cells even in the presence of sufficient oxygen (Warburg effect). Lactate-derived lactylation of histone lysine residues has recently emerged as an epigenetic modification that directly induces pro-inflammatory gene transcription in LPS and IFNγ-challenged macrophages. In fact, it is now well-established that glycolysis drives pro-inflammatory M1-macrophage polarization, even in the context of HIV infection. This is in agreement with in vivo and in vitro data on pyruvic acid. While no previous studies have evaluated the impact of plasma metabolic alterations in ATI, one study observed a glycolytic plasma profile in transient HIV elite controllers (TECs) compared to persistent elite controllers (PECs). Moreover, glutamic acid was shown to be elevated in PECs compared to TECs, corresponding to the observation that glutamate metabolism associated with delayed time to HIV rebound. In totality, a global Warburg phenotype has now emerged as a classic manifestation of HIV infection. Thus, the plasma metabolite signatures observed are likely a snapshot of the global and intrinsic cellular metabolic flux that occurs during ATI in individual patients.

In addition to L-glutamic acid and pyruvic acid, other intriguing plasma metabolites emerged from the analysis of the Philadelphia cohort. Among the plasma markers associated with delayed viral rebound is ethylmalonic acid. Ethylmalonic acid is central in the metabolism of butyrate, a short-chain fatty acid produced by the gut microbiota and known for its anti-inflammatory effects. Another group of metabolites, consisting of indole-3-pyruvic acid, indole-3-lactic acid, 3-indoxyl sulphate, and 2-oxindole, characterized accelerated rebound and may reflect a biochemical manifestation of dysbiosis of gut bacteria resulting in tryptophan catabolism. Indeed the tryptophan metabolic pathway was highlighted as one of the main metabolic pathways associated with accelerated viral rebound.

Although it was not mechanistically interrogated, a positive association between plasma indoleamine 2,3-dioxygenase (IDO) activity (an immunoregulatory enzyme that metabolizes tryptophan) and total HIV DNA in peripheral blood has been established. Impaired intestinal barrier integrity is a classical feature of HIV infection, characterized by dysbiosis and increased microbial by-products that drive systemic and mucosal inflammation. Microbes with the capacity to catabolize tryptophan have been linked to adverse HIV disease progression, at least in part in due to induction of IDO1 that interferes with Th17/Treg balance in the periphery and gut. The data highlight previously unrecognized interactions between the gut microbiome, its metabolic activity, and HIV persistence. Understanding these potential multi-nodal complex relationships during ART and post ATI warrants further investigation.

Similar to metabolites, glycans on glycoproteins are bioactive molecules and can play significant roles in mediating immunological functions. For example, antibody glycans can alter an antibody's Fc-mediated innate immune functions, including antibody-dependent cell-mediated cytotoxicity (ADCC) and several pro- and anti-inflammatory activities. Among glycans on antibodies, the presence of core fucose results in a weaker binding to Fcγ receptor IIIA and reduces ADCC. The same occurs with terminal sialic acid, which reduces ADCC. On the other hand, terminal galactose induces ADCC. In three independent geographically-distinct cohorts, two studied in a previous pilot study and the ACTG cohort studied in the current study, it was observed that a significant association between pre-ATI levels of the digalactosylated non-fucosylated non-sialylated glycan, G2, and delayed viral rebound. G2 is the only IgG glycan trait that is terminally galactosylated, non-fucosylated, and non-sialylated (FIG. 12) which is compatible with higher ADCC activity. On the other hand, sialylated glycan traits A2 on IgG and A3G3S3 on total plasma N-glycome associated with accelerated rebound and lower probability of viral remission. Similar to the pilot study, a link between plasma levels of N-acetyl-glucosamine (GlcNAc) and delayed viral rebound was observed. GlcNAc has been reported to have an anti-inflammatory impact during several inflammatory diseases. GlcNAc can influence cell signaling and modulate the expression of several transcription factors including NFκB. There are potential direct links between the impact of these glycans on innate immune functions and inflammation, and how this affects HIV control during ART.

Glycoproteins can also be shed from cells in different organs; therefore, their characteristics can reflect these cells' functions. Glycans on the cell surface are involved in signaling cascades controlling several cellular processes. It is not clear how the higher pre-ATI levels of plasma fucose, which associate with both delayed viral rebound and higher likelihood for PTC status post-ATI, can directly impact viral control during ATI. Nor it is clear how the higher pre-ATI levels of plasma GalNAc-containing glycans (T/Tn antigens), which associate with both accelerated viral rebound and lower likelihood for PTC status, can directly impact viral control during ATI.

However, these higher levels might reflect differential levels of these glycans on cells in different organs. For example, T-antigens (tumor-associated antigen) and Tn antigen are O-glycans that are truncated and have incomplete glycosylation. These antigens are not usually expressed on the surface of normal cells but are commonly present in cancerous cells and have been used as tumor markers. These truncated O GalNAc-containing glycans expressed on some normal immune cells (such as T cells) are ligands of the macrophage galactose type lectin (MGL) that is expressed on activated antigen presenting cells (APCs) such as macrophages and dendritic cells (DCs). MGL interacts with GalNAc-containing glycans on T cells significantly reduce CD45 phosphatase activity and inhibit T cell receptor signaling, resulting in T cell dysfunction. The data show that higher levels of these antigens in plasma ae associated with accelerated rebound and lower likelihood of viral remission and raise the question of whether these glycan levels reflect an immunosuppressive environment in NCs and those who rebound fast.

The following examples disclose specific embodiments of the methods and compositions of the present invention and should be construed to encompass any and all variations that become evident as a result of the teachings provided herein.

### EXAMPLES

### Example 1: Methods

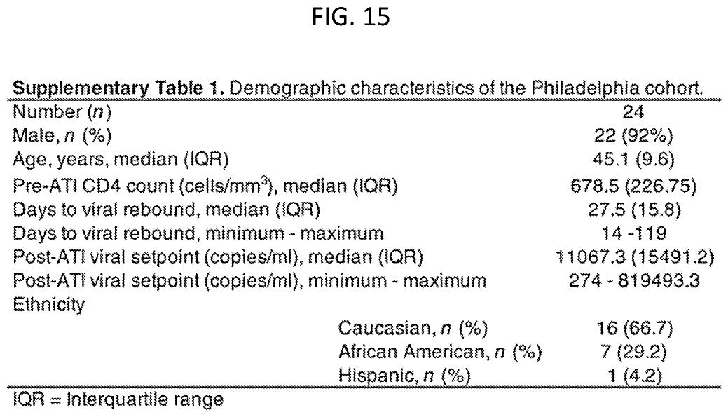

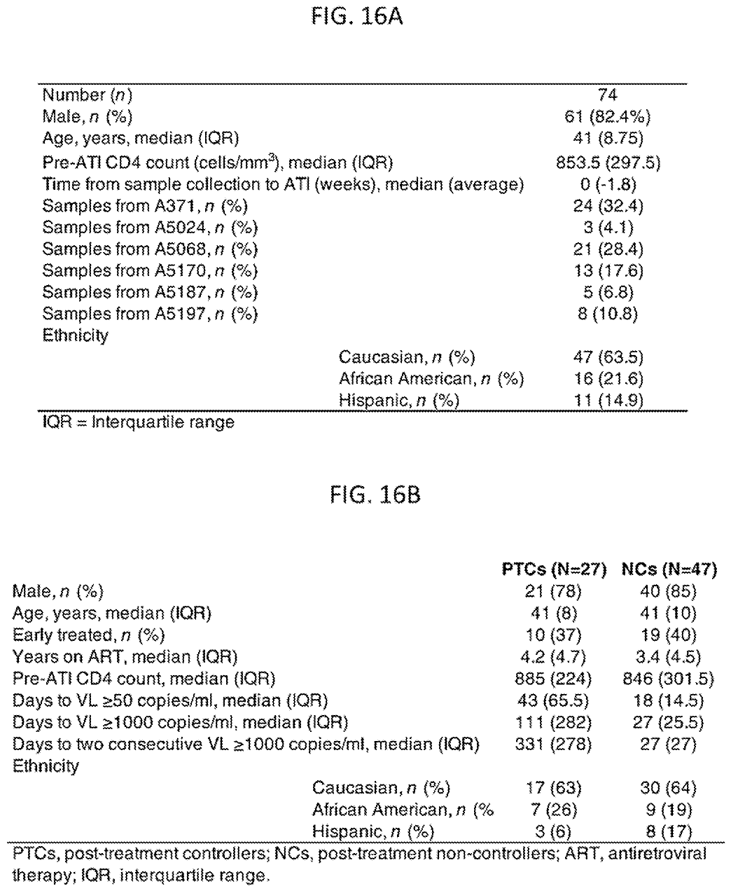

Study cohorts. Analyses were performed from banked plasma samples of two different cohorts that underwent analytical treatment interruption (ATI): (1) Philadelphia Cohort and (2) ACTG cohort. In the Philadelphia cohort, 24 HIV+ individuals on suppressive ART underwent an open-ended ATI without concurrent immunomodulatory agents. This cohort had a wide distribution of viral rebound times (14 to 119 days; median=28; FIG. 15). The ACTG cohort combined 74 HIV-infected ART-suppressed participants who underwent ATI from six ACTG ATI studies (ACTG 371, A5024, A5068, A5170, A5187, and A5197). 27 of these 74 individuals exhibited a PTC phenotype post-ATI, i.e. these individuals remained off ART for ≥24 weeks post-treatment interruption, sustained virologic control for at least 24 weeks, maintained viral load (VL)≤400 copies for at least ⅔ of time points, had plasma drug level testing performed, and had no evidence of spontaneous control pre-ART. The remaining 47 cohort members were non-controllers (NCs) who exhibited virologic rebound before meeting PTC criteria. These two groups were matched for gender, age, % treated at the early stage of HIV infection, ART duration, pre-ATI CD4 count, and ethnicity, as shown in FIG. 16. All analyses were performed on samples collected immediately before ATI in both cohorts.

Plasma untargeted metabolomics analysis. Metabolomics analysis was performed as described previously. Briefly, polar metabolites were extracted from plasma samples with 80% methanol. A quality control (QC) sample was generated by pooling equal volumes of all samples and was injected periodically during the sequence of LC-MS runs. LC-MS was performed on a Thermo Scientific Q Exactive HF-X mass spectrometer with HESI II probe and Vanquish Horizon UHPLC system. Hydrophilic interaction liquid chromatography was performed at 0.2 ml/min on a ZIC-pHILIC column (2.1 mm×150 mm, EMD Millipore) at 45° C. Solvent A was 20 mM ammonium carbonate, 0.1% ammonium hydroxide, pH 9.2, and solvent B was acetonitrile. The gradient was 85% B for 2 min, 85% B to 20% B over 15 min, 20% B to 85% B over 0.1 min, and 85% B for 8.9 min. All samples were analyzed by full MS with polarity switching. The QC sample was also analyzed by data-dependent MS/MS with separate runs for positive and negative ion modes. Full MS scans were acquired at 120,000 resolution with a scan range of 65-975 m/z. Data-dependent MS/MS scans were acquired for the top 10 highest intensity ions at 15,000 resolution with an isolation width of 1.0 m/z and stepped normalized collision energy of 20-40-60. Data analysis was performed using Compound Discoverer 3.1 (ThermoFisher Scientific). Metabolites were identified with high confidence by accurate mass and retention time using an in-house database generated from pure standards. Additional metabolites were tentatively identified by querying the mzCloud database (mzCloud.org) with MS/MS spectral data and selecting matches with scores of 50 or greater. Metabolite quantification used peak areas from full MS runs and were corrected based on the periodic QC runs. Peak areas from samples of the ACTG study were normalized to the summed area for identified metabolites in each sample.

In-vitro examination of the impact of L-glutamic acid on latent HIV reactivation. J-Lat cells were used as model of HIV latency. J-Lat cells harbor latent, transcriptionally-competent HIV provirus that encodes green fluorescent protein (GFP) as an indicator of viral reactivation. Levels of latent HIV transcription after stimulation can be measured using flow cytometry. L-glutamic acid was purchased from Sigma (catalog #49449-100G) and was dissolved in cell-culture compatible HCl solution (Sigma catalog #H9892-100ML) according to the manufacture's guidelines. Cells from different clones of J-Lat (5A8 and 10.6) were cultured at 1×10⁶ cells/ml in cultured in R10 media (RPMI 1640 medium supplemented with 10% fetal bovine serum (FBS), L-glutamine (2 mM), penicillin (50 U/ml), and streptomycin (50 mg/ml)), and were stimulated with PMA/ionomycin (16 nM/500 nM—Sigma catalog #P8139/catalog #I0634-1MG, respectively) or ImmunoCult Human CD3/CD28 T Cell Activator (Stem cell catalog #10971), or TNFα (10 ng/ml; Stem Cell catalog #78068.1) in the presence of HCl solution as a control. J-Lat cells were also treated with L-glutamic acid (5 mM) in the presence or absence of the above stimulators. After 24 hours, cells were stained with live/dead marker (Thermo catalog #L34966) and GFP Mean Fluorescence intensity (MFI) was measured by LSR II flow cytometer and FACSDiva software.

In-vitro examination of the impact of L-glutamic acid and pyruvate on myeloid inflammation. THP1 cells were plated in 24-well plates at a density of 7×10⁵ cells per well. To differentiate them into macrophages-like, 100 nM of PMA (Sigma catalog #P8139) was added and incubated for 72 hours. After incubation, media was aspirated, and each well was gently washed twice with R10 media. Cells were then rested for 24 hours on R10 media without PMA. After 24 hours, cells were washed again with serum-free RPMI 1640 media and kept on this media for the rest of experiment. Macrophage-like THP1 cells were pre-incubated with L-glutamic acid (5 mM) or Sodium Pyruvate solution (2 mM, Sigma catalog #S8636-100 ml) for 2 hours before stimulating with Escherichia coli serotype O127:B8 LPS (50 ng/ml; Sigma catalog #L3129-10MG) and IFNγ (10 ng/ml; R&D Systems catalog #285-IF-100, respectively). After 5 hours of incubation with LPS/IFNγ, culture supernatants were collected for cytokine quantitation. Supernatant levels of IL-10, IL-12p70, IL-13, IL-1β, IL-2, IL-4, IL-6, and IL-8 were determined using U-PLEX Proinflam Combo 1 (Meso Scale Diagnostic #K15049k-1) according to manufacture. Levels of TNF-α were quantified using DuoSet ELISA kits (R&D Systems catalog #DY210-05).

IgG isolation. Bulk IgG was purified from 50p plasma using Pierce Protein G Spin Plates (Thermo Fisher catalog #45204). IgG purity was confirmed by SDS gel.

N-glycan analysis using capillary electrophoresis. For both plasma and bulk IgG, N-glycans were released using peptide-N-glycosidase F (PNGase F) and labeled with 8-aminopyrene-1,3,6-trisulfonic acid (APTS) using the GlycanAssure APTS Kit (Thermo Fisher cat. A33952), following the manufacturer's protocol. Labeled N-glycans were analyzed using the 3500 Genetic Analyzer capillary electrophoresis system. IgG N-glycan samples were separated into 19 peaks and total plasma N-glycans into 24 peaks. Relative abundance of N-glycan structures was quantified by calculating the area under the curve of each glycan structure divided by the total glycans using the Applied Biosystems GlycanAssure Data Analysis Software Version 2.0.

Glycan analysis using lectin array. To profile the plasma total glycome, the lectin microarray was used as it enables analysis of multiple glycan structures; it employs a panel of 45 immobilized lectins with known glycan-binding specificity. Plasma proteins were labeled with Cy3 and hybridized to the lectin microarray. The resulting chips were scanned for fluorescence intensity on each lectin-coated spot using an evanescent-field fluorescence scanner GlycoStation Reader (GlycoTechnica Ltd.), and data were normalized using the global normalization method.

Quantification ofHIVDNA and CA-RNA. Total RNA and DNA were isolated from CD4+ T cells using the AllPrep DNA/RNA Mini Kit (Qiagen). Unspliced cell-associated HIV RNA levels and Total HIV DNA levels were quantified using a real-time PCR approach with primers/probes targeting conserved regions of HIV LTR/gag. Cell numbers were quantified by qPCR measurements of CCR5 DNA copy numbers.

### Example 2: Characteristics of Study Cohorts

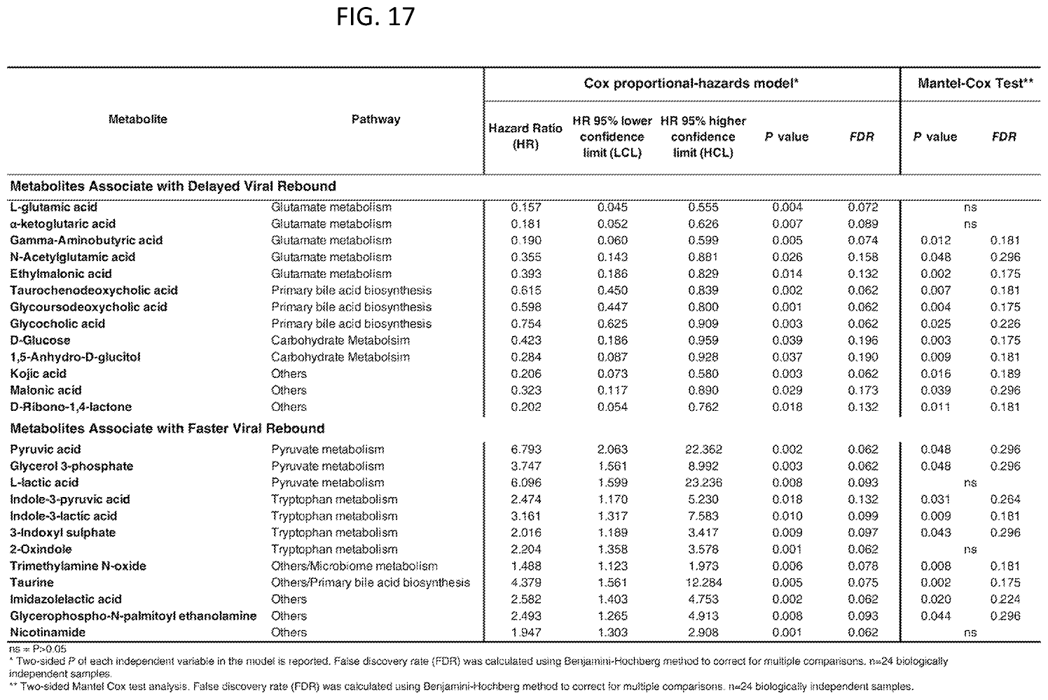

In this study, two ATI cohorts were employed: (1) The Philadelphia cohort: a group of 24 HIV-infected individuals on suppressive ART who underwent an open-ended ATI. This cohort had a wide distribution of viral rebound times (14-119 days; median=28; FIG. 15). Importantly, this cohort underwent ATI without concurrent immunomodulatory agents that might confound the signatures at the initial phase of the study. (2) The ACTG cohort: a cohort that combined 74 participants from six ACTG ATI studies (ACTG 37124, A502425, A506826, A517027, A518728, and A519729), which tested the efficacy of different HIV vaccines and/or immunotherapies. These six ATI studies included 600 participants and identified 27 PTCs among their participants. The ACTG cohort included all 27 PTCs and 47 matched non-controllers (NCs) from the same studies. The definition of post-treatment control was: remaining off ART for ≥24 weeks post-ATI with viral load (VL)≤400 copies for at least ⅔ of time points; no ART in the plasma; and no evidence of spontaneous control pre-ART. The 47 NCs rebounded before meeting PTC criteria, The PTC and NC groups within the ACTG cohort are matched for sex, age, ethnicity, percent treated during early infection, ART duration, and pre-ATI CD4 count (FIG. 16A, FIG. 16B, and FIG. 17). Notably, the individuals in the ACTG cohort had participated in clinical trials where they had received, or not, different HIV vaccines and/or immunotherapies. This important feature of this cohort allows for identifying/validating markers that predict duration and probability of viral remission independent of potential interventions. Metabolic and glycomic analyses were performed on samples collected at one timepoint, shortly before the ATI.

### Example 3: Elevated Pre-ATI Levels of Plasma Markers of Glutamate and Bile Acid Metabolism Associate with Delayed Viral Rebound in the Philadelphia Cohort

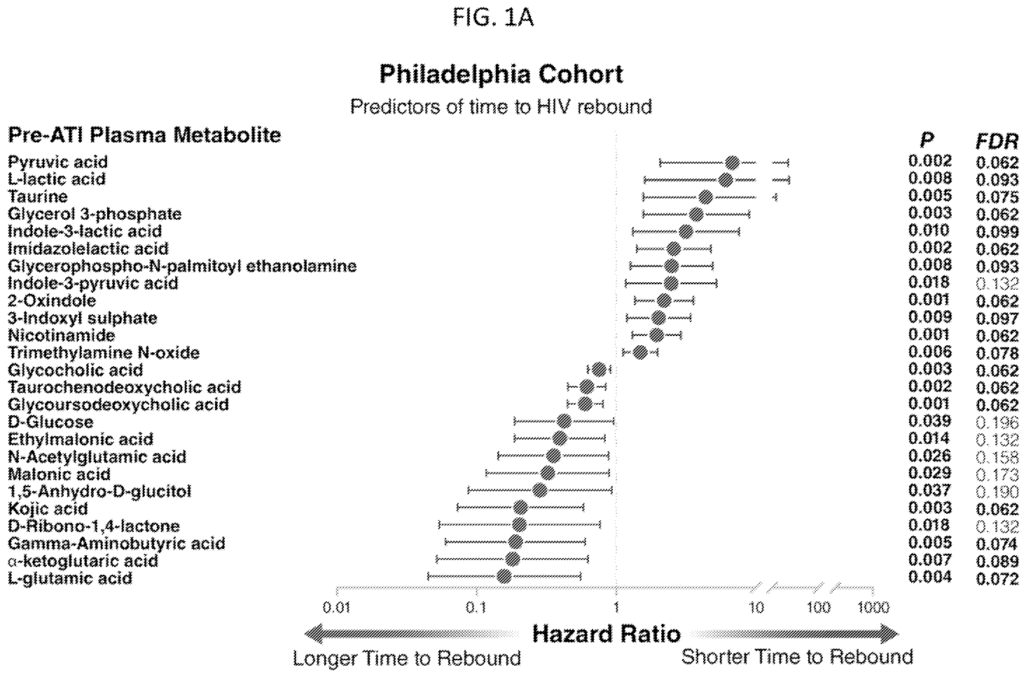

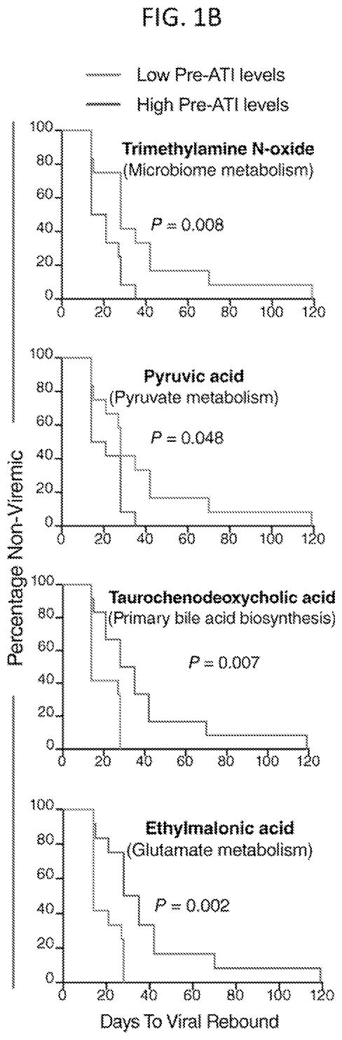

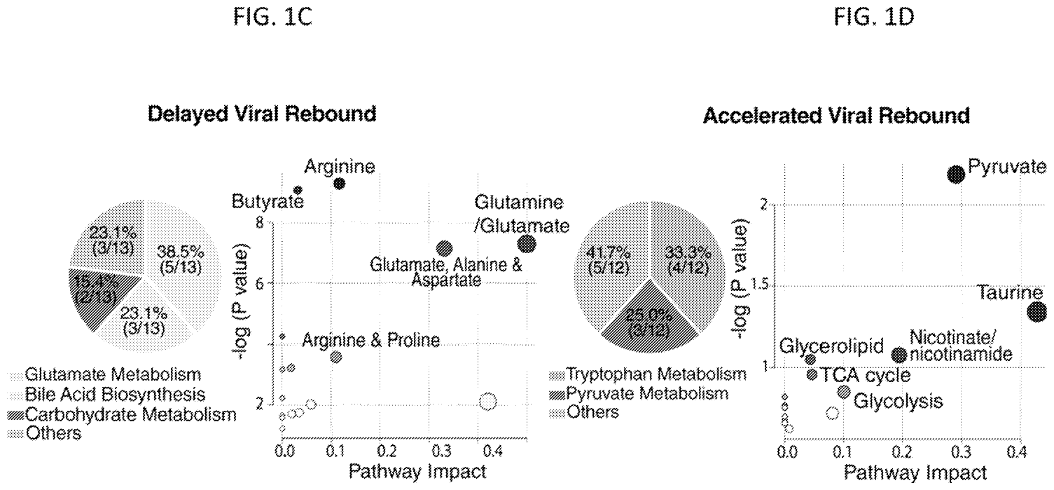

We first aimed to examine the utility of plasma metabolites as biomarkers of time-to-HIV-rebound after ART cessation. Towards this goal, we measured levels of plasma metabolites from the Philadelphia cohort. Using an untargeted mass spectrometry (MS)-based metabolomics analysis, we identified a total of 179 metabolites in the plasma samples collected immediately before the ATI. Then, we applied the Cox proportional-hazards model to identify metabolomic signatures of time-to-viral-rebound. As shown in FIG. 1A, higher pre-ATI levels of 13 plasma metabolites were significantly associated with a longer time-to-viral-rebound with P<0.05 and false discovery rate (FDR)<20%. In contrast, higher pre-ATI levels of 12 plasma metabolites were significantly associated with a shorter time-to-viral-rebound. When participants were separated into low or high groups, based on the median of each of the 25 metabolic markers, the pre-ATI levels of 20 of 25 metabolites significantly indicated hazards of viral-rebound over time using the Mantel-Cox test (FIG. 1B and FIG. 17).

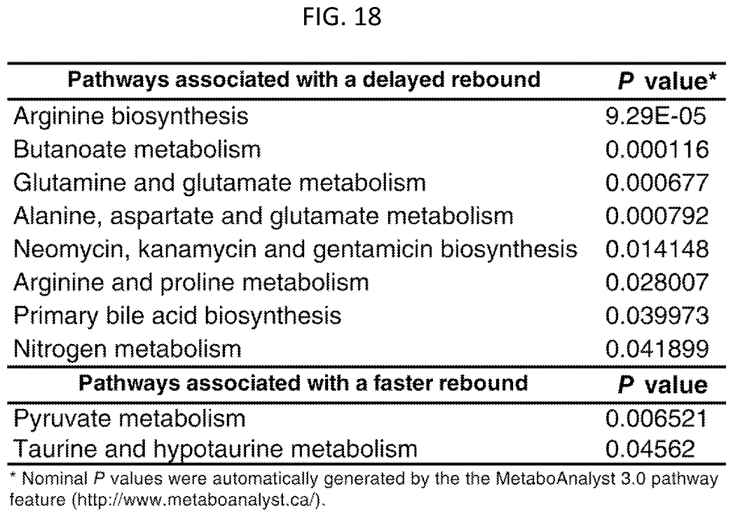

We next sought to determine if the 25 metabolites that associated with time-to-viral-rebound shared similar metabolic pathways. Multi-analysis combining KEGG and the STRING Interaction Network (focusing on metabolite-associated enzymatic interactions) revealed that most of the 13 metabolites whose pre-ATI levels associated with a longer time-to-viral-rebound belonged to two major metabolic pathways. Specifically, five metabolites lay within the anti-inflammatory glutamate/tricarboxylic acid (TCA) cycle pathway, and three were intermediates within the primary bile acid biosynthesis pathway (FIG. 1C). Confirmatory analysis on these 13 metabolites using the MetaboAnalyst 3.0 pathway feature showed enrichment in glutamate metabolism (P=0.00068) and the bile acid biosynthesis pathway (P=0.0399) (FIG. 1C and FIG. 18).

### Example 4: Elevated Pre-ATI Levels of Plasma Markers of Pyruvate and Tryptophan Metabolism Associate with Accelerated Viral Rebound in The Philadelphia Cohort

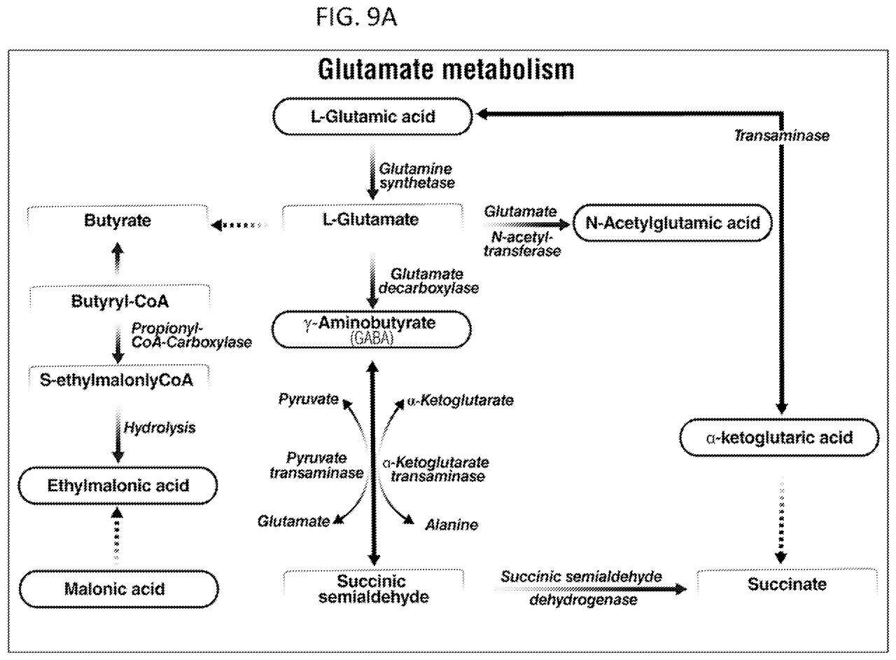

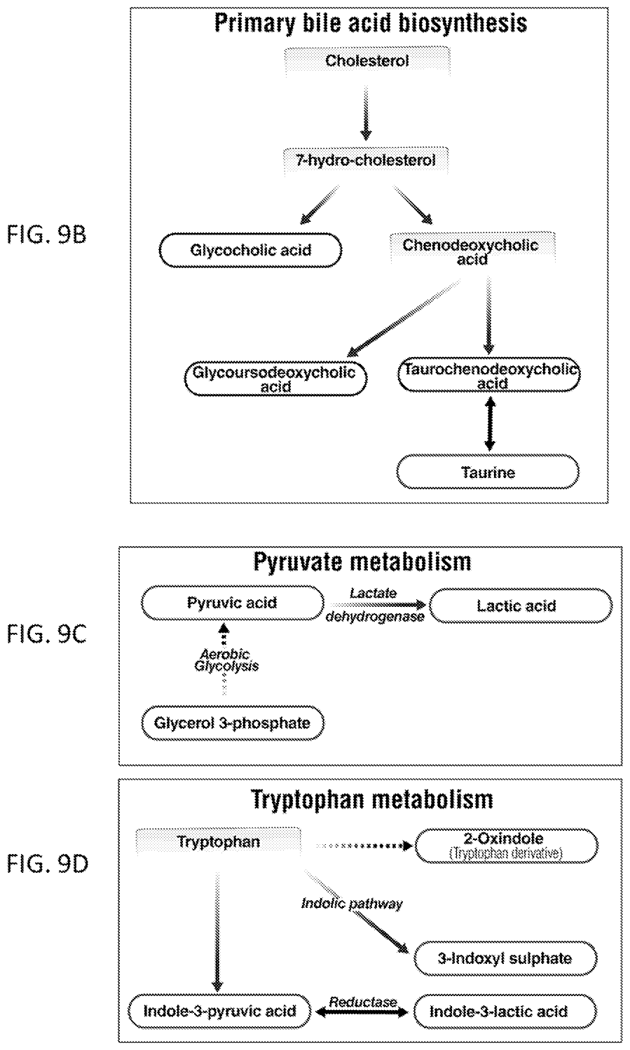

Multi-analysis of the 12 metabolites whose pre-ATI levels associated with shorter time-to-viral-rebound showed four intermediates in the tryptophan metabolism pathway and three that are central players in the pro-inflammatory pyruvate pathway (FIG. 1D). These observations were confirmed for the 12 metabolites using MetaboAnalyst 3.0, which demonstrated enrichment in pyruvate metabolism (P=0.0065) (FIG. 1D and FIG. 18). The roles of the 25 metabolites within the glutamate, bile acids, tryptophan, and pyruvate pathways are graphically illustrated in FIG. 9A-FIG. 9D. These data reveal a previously undiscovered class of plasma metabolic biomarkers that are associated with time-to-viral rebound post-ATI. They further demonstrate that these biomarkers belong to a specific set of metabolic pathways that may play a previously unrecognized role in HIV control.

### Example 5: L-Glutamic Acid and Pyruvic Acid Modulate Latent HIV Reactivation and/or Macrophage Inflammation In Vitro

Among the top candidate metabolic biomarkers from FIG. 1A-FIG. 1D are L-glutamic acid (glutamate metabolism) and pyruvic acid (pyruvate metabolism). The higher pre-ATI levels of L-glutamic acid and pyruvic acid were associated with a longer or a shorter time-to-viral-rebound, respectively. These two metabolites can impact inflammation in opposing directions. Glutamate controls the anti-inflammatory TCA cycle through its conversion by glutamate dehydrogenase to α-ketoglutarate, whereas pyruvate is centrally positioned within the pro-inflammatory glycolytic pathway. We therefore sought to determine if these two metabolites had a direct, functional impact on latent HIV transcription and/or myeloid inflammation.

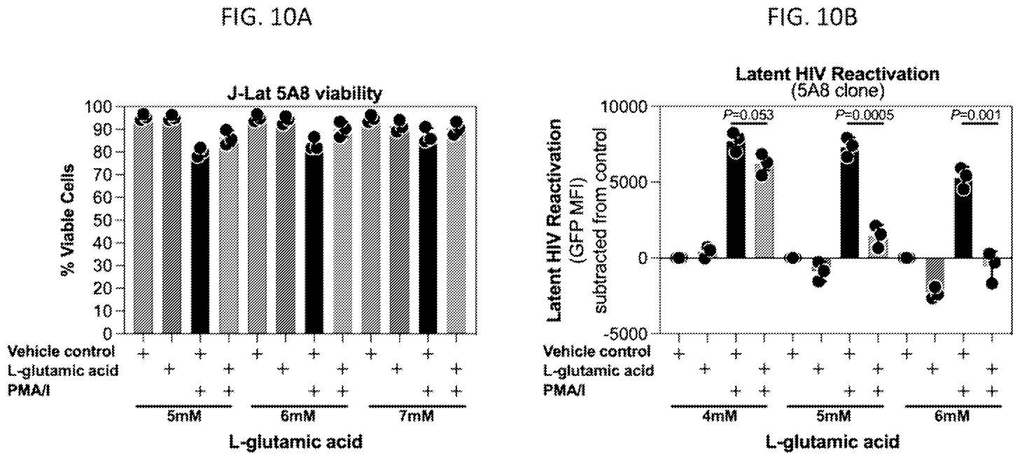

We first assessed the impact of each of these two metabolites on latent HIV reactivation using the established “J-Lat” model of HIV latency. J-Lat cells harbor a latent, transcriptionally competent HIV provirus that encodes green fluorescent protein (GFP) as an indicator of reactivation (FIG. 2A). There are several clones of the J-Lat model with different characteristics, including the type of stimulation to which they respond. For example, the 5A8 clone is the only J-Lat clone responsive to αCD3/αCD28 stimulation. We examined the impact of L-glutamic acid and pyruvate on latent HIV reactivation using two J-Lat clones, 5A8 and 10.6. L-glutamic acid significantly inhibited the ability of phorbol-12-myristate-13-acetate (PMA)/ionomycin or αCD3/αCD28 to reactivate latent HIV in clone 5A8 without impacting viability, compared to stimuli alone controls (FIG. 2B). L-glutamic acid also inhibited the ability of PMA/ionomycin or TNFα to reactivate latent HIV in clone 10.6 without impacting viability, compared to stimuli alone controls (FIG. 2C). Finally, to test the impact of glutamine in the RPMI media on the results, J-Lat 5A8 cells were cultured in glutamine-free media and treated with L-glutamic acid at different doses, in the presence or absence of PMA/I. As shown in FIG. 10A and FIG. 10B, L-glutamic acid inhibited the ability of PMA/I to reactivate the 5A8 J-Lat clone in a dose-dependent manner in glutamine-free media. These data demonstrate that a plasma metabolite, L-glutamic acid, can inhibit latent viral reactivation, consistent with the observation that pre-ATI levels of L-glutamic acid predicted a longer time-to-viral-rebound.

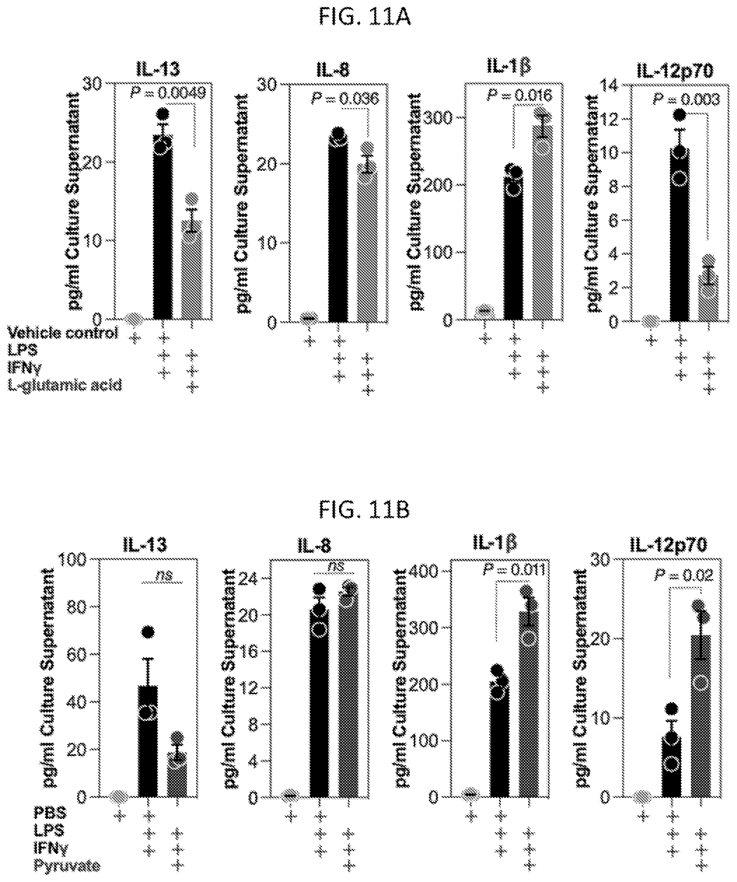

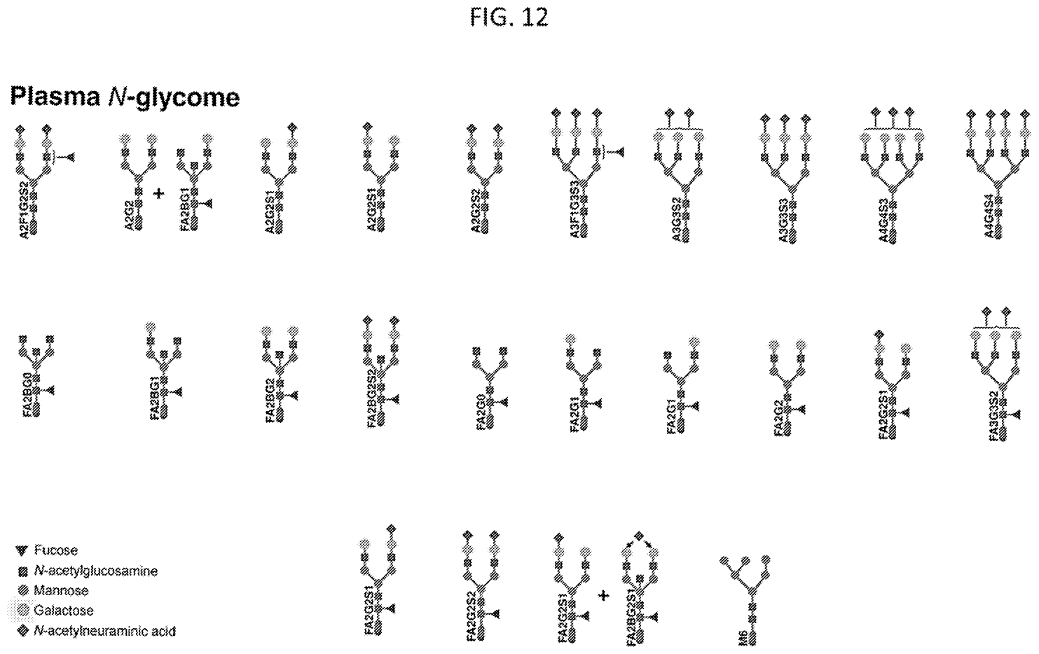

Beyond the direct impact on latent viral reactivation, plasma metabolites may exert effects on myeloid inflammation, and such effects may underlie HIV control during ATI. This possibility was tested by examining the effects of L-glutamic acid and pyruvate on lipopolysaccharides (LPS)-mediated secretion of pro-inflammatory cytokines from THP-1 derived macrophage-like cells. These cells are characterized by high basal glycolytic activity, which closely reflects the Warburg-like phenotype observed in HIV-infected individuals37. They also exhibit similar inflammatory responses as primary cells under similar in vitro conditions38. Cells were treated with L-glutamic acid, pyruvate, or appropriate controls for 2 h before stimulating with LPS and IFNγ for 5 h (FIG. 2D). L-glutamic acid inhibited LPS/IFNγ-mediated production of pro-inflammatory cytokines such as IL-6 and TNFα (FIG. 2E; other cytokines are shown in FIG. 11A). Consistently, L-glutamic acid also increased production of anti-inflammatory IL-10 (FIG. 2E). Conversely, pyruvate increased IL-6 and TNFα secretion (FIG. 2F; other cytokines are shown in FIG. 11B). These data demonstrate not only that some metabolites associate with time-to-viral-rebound, but also that there is a plausible, functionally significant link between these metabolic biomarkers and viral control during and following ATI.

### Example 6: Pre-ATI Plasma Glycomic and Metabolic Biomarkers Associate with Time-to-Viral-Rebound in the ACTA Cohort

A pilot study showed that pre-ATI levels of a specific set of glycans predicted a longer time-to-viral rebound after ART discontinuation (Giron et al. AIDS 34, 681-686 (2020)). However, this small pilot study did not correct for confounders such as age, sex, and nadir CD4 count on viral rebound. We hypothesized that a set of plasma glycans and metabolites we identified in that pilot study, as well as in the results shown in FIG. 1A-FIG. 1D, can predict time-to-viral-rebound and/or probability-of-viral-rebound, even after adjusting for potential demographic and clinical confounders. For this analysis of a larger validation cohort, we analyzed samples from the ACTG cohort.

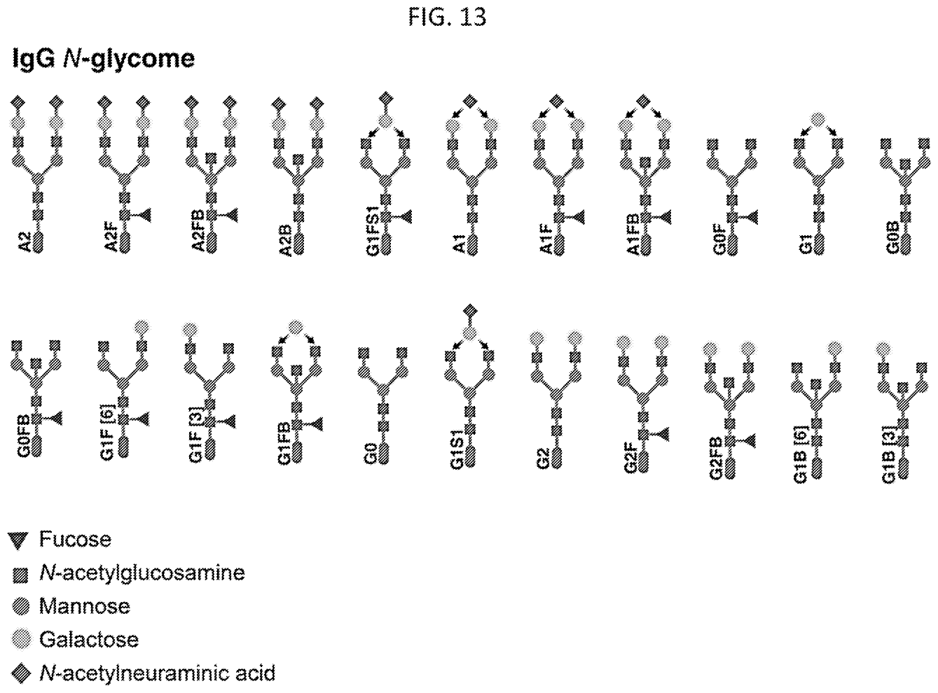

We analyzed the plasma metabolome of samples collected from this cohort before ATI. A total of 226 metabolites were identified using MS-based metabolomics analysis. In addition, we analyzed the plasma glycome of the same samples by applying two different glycomic technologies. First, we used capillary electrophoresis to identify the N-linked glycans of total plasma glycoproteins (we identified 24 glycan structures, their names and structures are listed in FIG. 12) and of isolated plasma IgG (we identified 22 glycan structures, their names and structures are listed in FIG. 13). Second, we used a 45-plex lectin microarray to identify total (N and O linked) glycans on plasma glycoproteins. The lectin microarray enables sensitive identification of multiple glycan structures by employing a panel of 45 immobilized lectins (glycan-binding proteins) with known glycan-binding specificity, resulting in a “glycan signature” for each sample (the 45 lectins and their glycan-binding specificities are listed in FIG. 19).

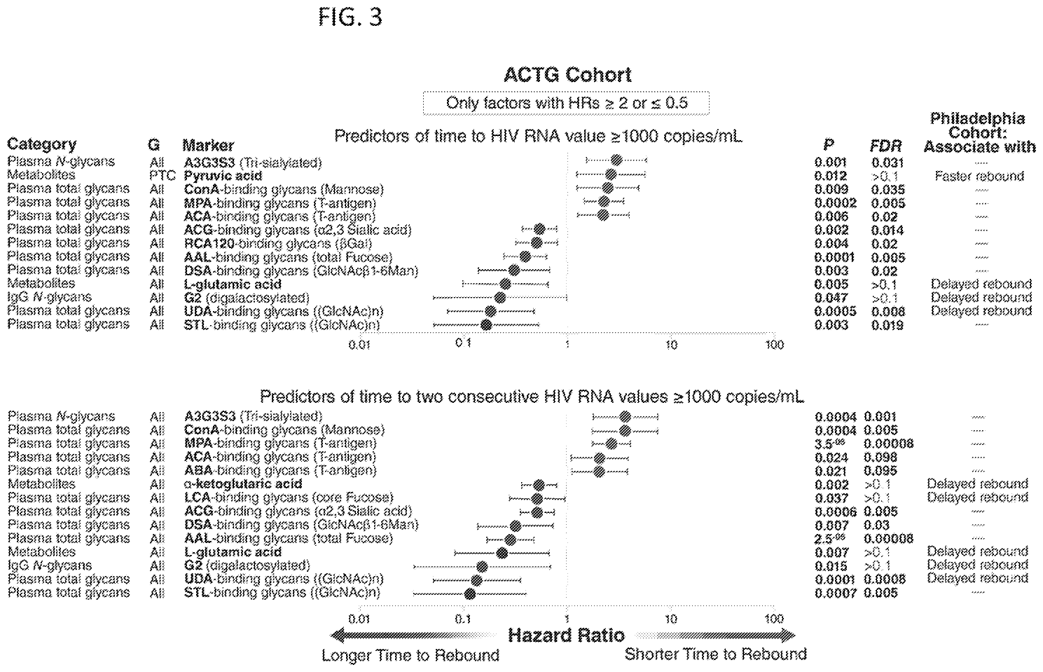

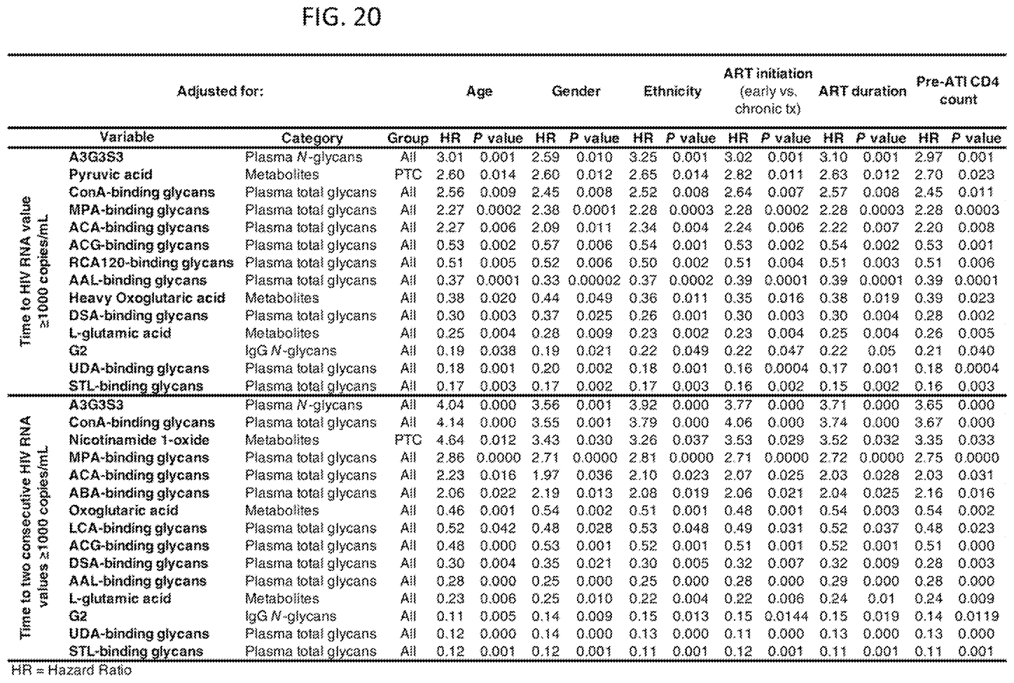

We used the Cox proportional-hazards model and a set of highly stringent criteria to identify sets of glycans or metabolites whose pre-ATI levels associate with either time to VL≥1000 copies/ml (FIG. 3, top panel) or time to two consecutive VL≥1000 copies/ml (FIG. 3, bottom panel). To ensure high stringency, we only considered markers with a hazard ratio (HR)≥2 or ≤0.5. We also only included markers with FDR<10%, or markers that had emerged from the Philadelphia cohort (FIG. 1A-FIG. 1D). Importantly, we only included markers that remained significant (P<0.05) after adjusting for age, sex, ethnicity, ART initiation (during early or chronic HIV infection), ART duration, or pre-ATI CD4 count (FIG. 20). These combined strict criteria identified a plasma signature that predicted shorter time-to-rebound to VL≥1000 copies/ml, comprising four glycan structures and one metabolite (FIG. 3). These five markers include the highly sialylated plasma N-glycan structure A3G3S3, GalNAc-containing glycans (also known as T-antigen; measured by binding to both MPA and ACA lectins), and the metabolite pyruvic acid. We also identified a signature that associated with a longer time-to-rebound to VL≥1000 copies/ml, comprising seven glycan structures and one metabolite, notably the digalactosylated G2 glycan structure on plasma bulk IgG, fucosylated glycans in plasma (binding to AAL lectin), GlcNac glycans in plasma (binding to DSA, UDA, and STL lectins), and the metabolite L-glutamic acid (FIG. 3, top panel).

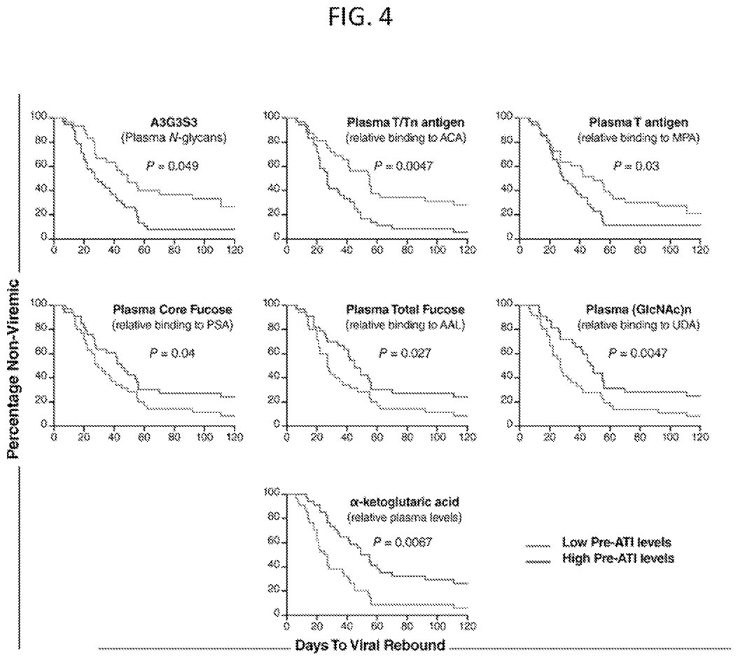

Turning to markers that associated with time to two consecutive VL≥1000 copies/ml, and applying the same strict criteria, we identified five glycomic markers whose pre-ATI levels associate with shorter time-to-rebound post-ATI, including A3G3S3 in plasma and T/Tn-antigens (binding to MPA, ACA, and ABA lectins) (FIG. 3, bottom panel). We also identified seven glycan structures and two metabolites whose pre-ATI levels predicted a longer time-to-rebound, including G2 glycan structure on bulk IgG, core fucosylated glycans (binding to LCA lectin) in plasma, total fucosylated glycans (binding to AAL lectin) in plasma, GlcNac glycans (binding to DSA, UDA, and STL lectins) in plasma, and the metabolites oxoglutaric acid (α-ketoglutaric acid) and L-glutamic acid (FIG. 3, bottom panel). The significance of several of these markers was also confirmed using the Mantel-Cox test in an independent analysis (FIG. 4). In sum, using stringent analysis criteria that also took into account potential confounders, we identified plasma glycomic/metabolomic signatures of time-to-viral-rebound after ART discontinuation in this independent, heterogeneous cohort of individuals who underwent ATI and received or not several different interventions before ATI.

### Example 7: Levels of Pre-ATI Plasma Glycomic and Metabolic Markers that Associate with Time-to-Viral-Rebound are Linked to Levels of Total, Intact, and Defective Cell-Associated HIV DNA as Well as Cell-Associated HIV RNA in the Blood

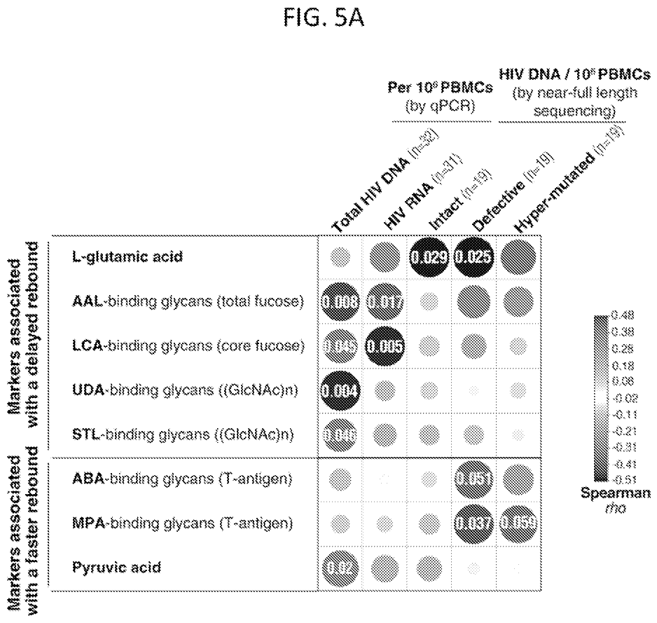

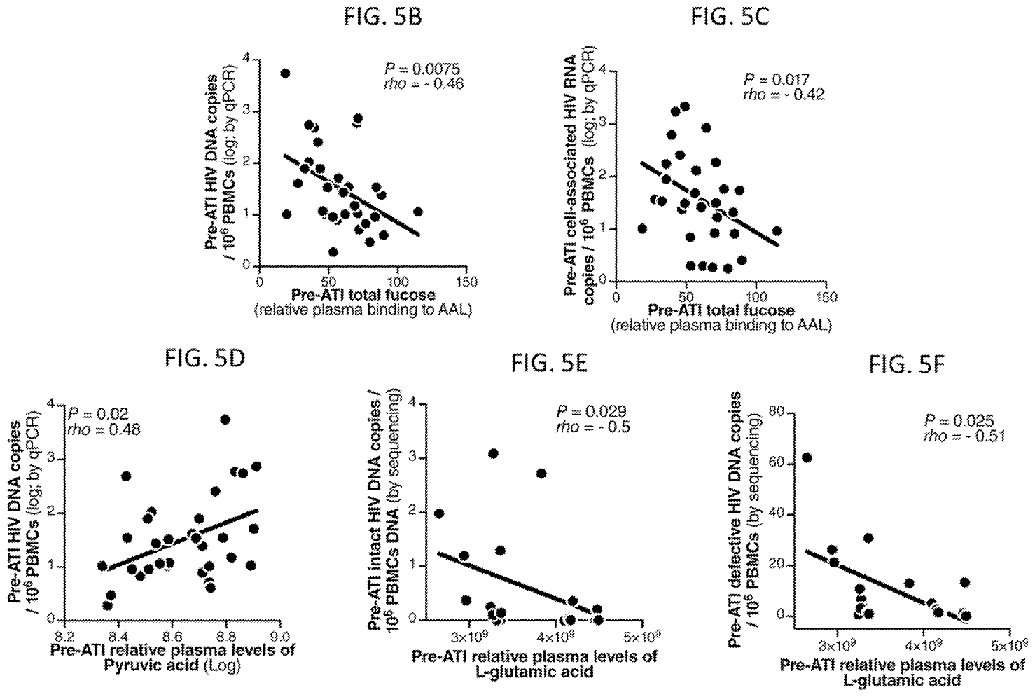

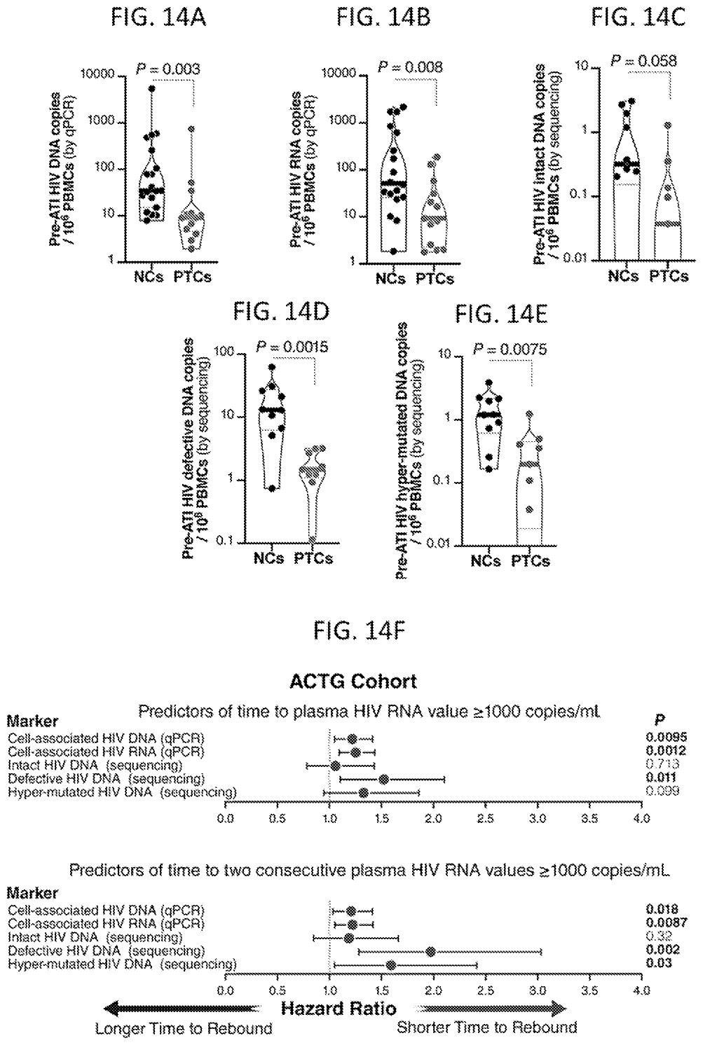

We next examined whether the plasma glycans and metabolites (FIG. 3) that associated with time-to-viral-rebound also reflected levels of virological markers of HIV persistence. We measured levels of peripheral blood mononuclear cell (PBMC)-associated total HIV DNA and HIV RNA by qPCR on a subset of 32 individuals from the ACTG cohort. Pre-ATI levels of cell-associated HIV DNA and RNA have been shown to correlate with time-to-viral-rebound in several previous studies. Indeed, levels of cell-associated HIV DNA and RNA were lower in PTCs compared to NCs and predicted time-to-viral-rebound using the Cox proportional-hazards model (FIG. 14A-FIG. 14F). When we examined the associations between these measures and the glycomic and metabolic markers, we found that pre-ATI levels of total fucose (binding to AAL lectin), which predicted delayed viral rebound, showed a significant inverse correlation with pre-ATI levels of cell-associated HIV DNA and RNA (FIG. 5A-FIG. 5C). Similarly, pre-ATI levels of core fucose (binding to LCA lectin), which also predicted delayed viral rebound, also showed an inverse correlation with pre-ATI levels of cell-associated HIV DNA and RNA (FIG. 5A). Furthermore, total levels of (GlcNAc)n (binding to UDA and STL lectins), which predicted delayed viral rebound, had an inverse correlation with levels of total HIV DNA (FIG. 5A). Noteworthy, levels of pyruvic acid, whose pre-ATI levels predicted accelerated viral rebound, had a significant positive correlation with pre-ATI levels of cell-associated HIV DNA (FIG. 5A, FIG. 5D).

The majority of HIV DNA harbor mutations and/or deletions, rendering them defective. Intact HIV proviruses can support viral transcription and translation; however, recently, it was also shown that some defective HIV proviruses can express viral RNA and proteins. We sought to examine the potential links between the plasma markers and levels of intact and defective HIV DNA. To do this, we took advantage of near-full length sequencing data that were recently generated on a subset of 19 individuals from this cohort (10 PTCs and 9 NCs). Within these 19 individuals, levels of intact, defective, and hypermutated HIV DNA were lower in PTCs compared to NCs, and levels of defective HIV DNA predicted time-to-viral-rebound using the Cox proportional-hazards model (FIG. 14-FIG. 14F). When we examined the associations between these measures and the glycomic and metabolic markers, we found that pre-ATI levels of L-glutamic acid, which predicted delayed viral rebound in both the Philadelphia and ACTG cohorts, showed a significant inverse correlation with pre-ATI levels of intact and defective (but not hypermutated) cell-associated HIV DNA (FIG. 5A, FIG. 5E, and FIG. 5F). In general, there was a trend for negative correlations between the pre-ATI levels of markers associated with a delayed viral rebound and the size of HIV reservoir (FIG. 5A). On the other hand, there was a trend for positive correlations between the pre-ATI levels of markers associated with a faster viral rebound and the size of HIV reservoir (FIG. 5A). These data provide more support for plausible connections between the discovered plasma markers and HIV persistence and control during ATI.

### Example 8: Multivariable Cox Model, Using Lasso Technique with Cross-Validation (CV), Selected Variables Whose Combination Predicts Time-to-Viral-Rebound

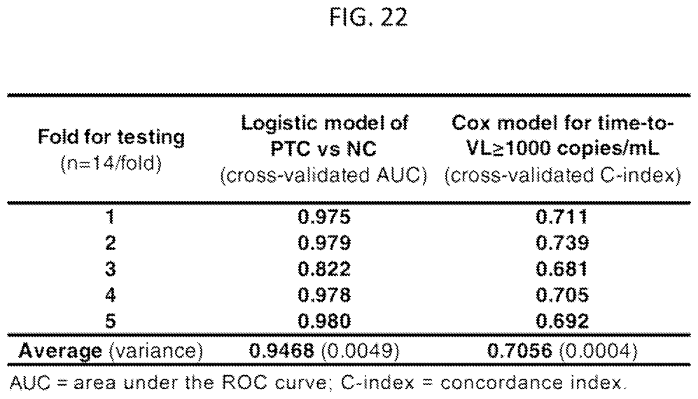

As a single marker would be highly unlikely to strongly predict these complex virological milestones, we next sought to apply a machine-learning algorithm to identify a smaller set of plasma biomarkers (from FIG. 3) that together can predict time to VL≥1000 copies/ml better than any of these biomarkers individually. The analysis considered biomarkers, both metabolites and/or glycan structures, that emerged as significant from the ACTG cohort (FIG. 3) and used data from ACTG samples with complete data sets (n=70; four samples did not have a complete dataset). The machine-learning algorithm, Lasso (least absolute shrinkage and selection operator) regularization, selected seven markers from the 13 that associated with time to VL≥1000 copies/ml (FIG. 3, top panel), whose predictive values are independent, and which, when combined, enhance the predictive ability of the signature compared to any marker alone (FIG. 21). Indeed, with the complete data from ACTG samples, a multivariable Cox regression model using these seven variables showed a concordance index (C-index) value of 74% (95% confidence interval: 68-80%), which is significantly higher than the C-index values obtained from Cox models using each variable individually (P<0.05; FIG. 21). Notably, these seven markers included four whose pre-ATI levels associated with accelerated rebound: A3G3S3, T-antigen (MPA and ACA lectins binding), and the metabolite pyruvic acid. The other three markers associated with delayed rebound: total fucose (AAL lectin binding), (GlcNAc)n (STL lectin binding), and the metabolite L-glutamic acid (FIG. 21). To be conservative, a more robust estimate of the model's performance was calculated using a 5-fold cross-validated model in the ACTG cohort, which resulted in an average C-index of 70.6% with a variance of 0.0004 (FIG. 22) Together, these data suggest that the multivariable model of combined plasma glycans and metabolites markers warrant further exploration for its capacity to predict time-to-viral-rebound in different settings.

### Example 9: Pre-ATI Plasma Glycomic and Metabolic Markers Distinguish Post-Treatment Controllers (PTCs) from Non-Controllers (NCs)

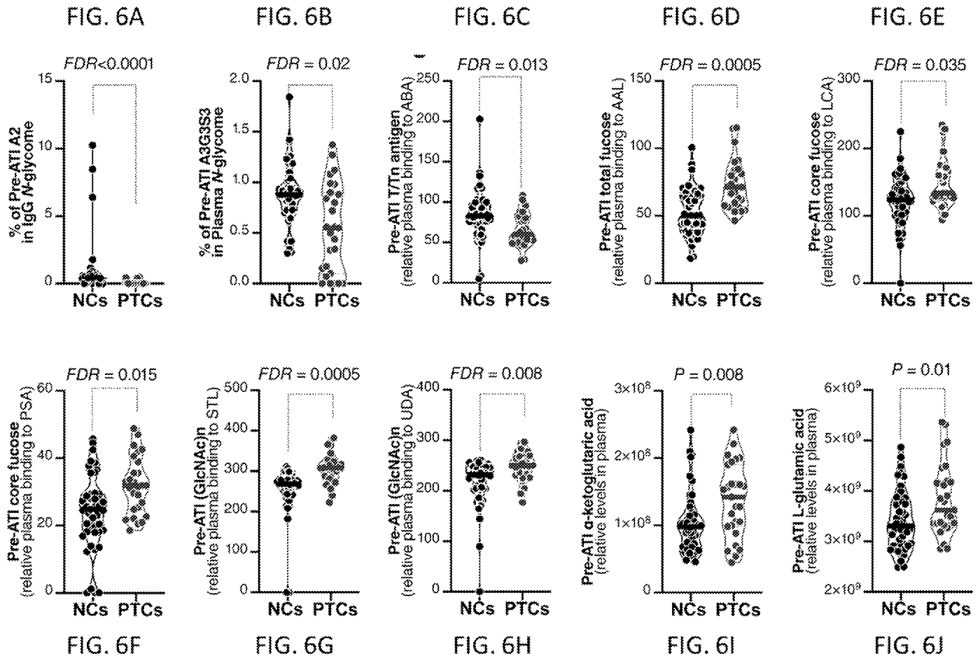

Examining the glycan structures and metabolites obtained from the ACTG cohort, we identified eight glycan structures whose pre-ATI levels were significantly different between PTCs and NCs with FDR<0.1 (FIG. 6A-FIG. 6H). Among these eight glycans structures, three exhibited lower levels in the plasma of PTCs compared to NCs (FDR<0.02), including the disialylated glycans, A2, in total IgG glycans; the highly sialylated glycans, A3G3S3, in plasma N-glycans; and T-antigen (binding to ABA lectin) (FIG. 6A-FIG. 6C); and five glycans were higher in PTCs compared to NCs (FDR≤0.035). These were total fucose (binding to AAL lectin), core fucose (binding to LCA and PSA lectins), and (GlcNac)n (binding to STL and UDA lectins (FIG. 6D-FIG. 6H).

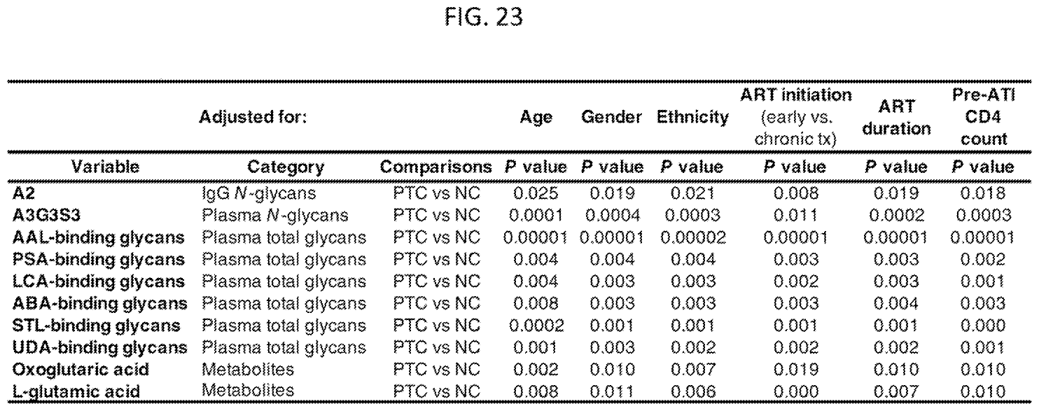

Examining metabolites, we found that pre-ATI levels of α-ketoglutaric acid and L-glutamic acid, both of which predicted delayed viral rebound, were higher in the plasma of PTCs compared to NCs (P<0.01, FIG. 6I, FIG. 6J). Importantly, this set of 10 markers contains only those markers whose levels remained different (P<0.05) between PTCs and NCs after adjusting for age, sex, ethnicity, ART initiation, ART duration, or pre-ATI CD4 count (FIG. 23). Together, these data suggest that a selective set of plasma glycans and metabolites can distinguish PTCs from NCs and may be used to predict the probability of viral rebound (i.e., the likelihood of PTC phenotype after ATI).

### Example 10: Multivariable Logistic Model, Using CV Lasso Technique, Selected Variables Whose Combination Predicts Risk of Viral Rebound

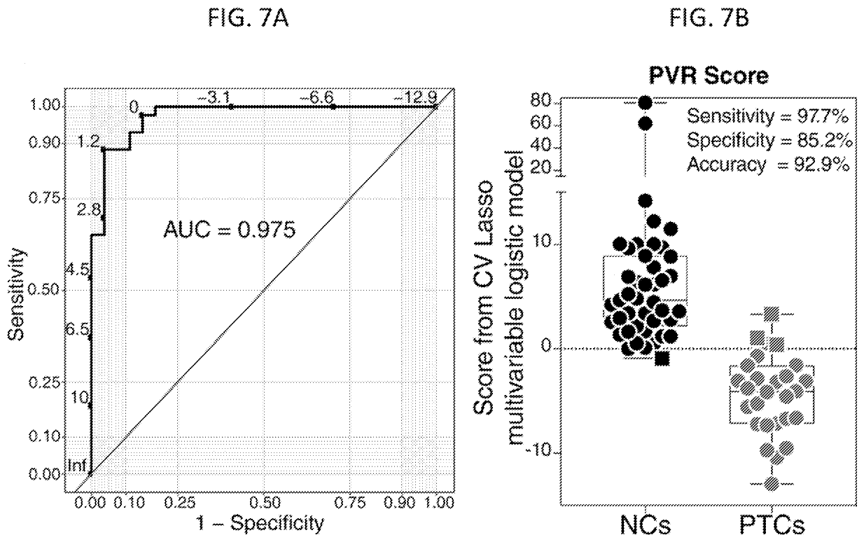

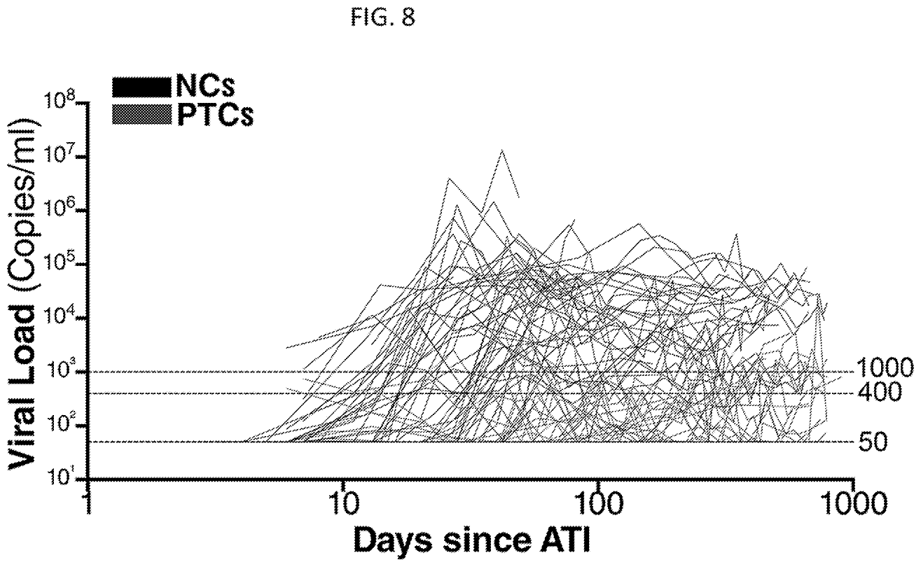

We next applied the Lasso regularization to select, from among the ten markers in FIG. 6A-FIG. 6J, a set of markers whose combined predictive utility is better than the predictive utility of any of these ten markers individually. The analysis used biomarkers that emerged as significant from the ACTG cohort (FIG. 6A-FIG. 6J) and only those samples with complete data sets (n=70). Lasso selected seven markers from the ten identified as able to distinguish PTCs from NCs whose predictive values are independent and combing them enhances the predictive ability of the signature compared to each of these markers alone (FIG. 21). Indeed, a multivariable logistic regression model using these seven variables showed an area under the ROC curve (AUC) value of 97.5% (FIG. 7a; 95% confidence interval: 94-100%), which is significantly higher than the AUC values obtained from logistic models using each variable individually (P<0.05; FIG. 21). A more robust estimate of the model's performance with a 5-fold cross-validated model in the ACTG cohort shows an average AUC of 94.7% with a variance of 0.0049 (FIG. 22). These seven markers included three whose pre-ATI levels are lower in PTCs compared to NCs, namely A2, A3G3S3, and T-antigen (ABA lectin binding), and four whose pre-ATI levels were higher in PTCs compared to NCs, namely total fucose (AAL lectin binding), core fucose (LCA lectin binding), (GlcNAc)n (STL lectin binding), and the metabolite L-glutamic acid (FIG. 21).

Next, a risk score predicting NC was estimated for each individual using the multivariable logistic model. We then examined the ability of these risk scores to classify PTCs and NCs from the ACTG cohort. As shown in FIG. 7B, the model correctly classified 97.7% of NCs (sensitivity) and 85.2% of PTCs (specificity) with an overall accuracy of 92.9%. This analysis highlights the potential utility of this risk score, estimated from the multivariable model and combining six plasma glycans and one metabolite, to predict the risk of NC post-ATI. This prediction can be used to select for ATI studies the individuals who are likely to achieve the PTC phenotype during HIV cure-focused clinical trials. In addition, the markers that are included in this model might also serve as windows into the mechanisms that contribute to the PTC phenotype.

### Example 11: Earlier Study—Plasma and Antibody Glycomic Biomarkers of Time to HIV Rebound and Viral Setpoint

HIV cure research urgently needs to identify a set of clinically accessible, pre-analytic treatment interruption (ATI) biomarkers that can predict time-to-viral-rebound and viral setpoint to improve the safety of ATI, accelerate the development of curative strategies, and provide biological clues into the molecular mechanistic underpinnings of HIV persistence mitigate the risk of ATI and accelerate development of a cure. We previously reported that galactosylated IgG glycans, G2, negatively correlate with cell-associated HIV DNA and RNA during antiretroviral therapy (ART). We hypothesized that this and other plasma glycomic traits can predict time-to-viral-rebound and viral setpoint upon ART cessation.

We hypothesized that G2 and other plasma glycomic traits may predict time-to-viral-rebound and viral setpoint upon ART cessation. To test this hypothesis, we profiled the plasma and bulk IgG glycomes from two geographically distinct cohorts of HIV-infected, ART-suppressed individuals who participated in ATI studies. We identified plasma and bulk IgG glycomic signatures, measured pre-ATI, that inform post-ATI time-to-viral-rebound and viral setpoints, and can serve, in the future, as novel, non-invasive predictive biomarkers.

A. Study cohorts—We profiled the circulating glycomic signatures (plasma and bulk IgG) from banked plasma samples from two geographically distinct cohorts: Philadelphia cohort—24 HIV-infected, ART-suppressed individuals who had participated in an open-ended ATI study without concurrent immunomodulatory agents. Johannesburg Cohort—23 HIV-infected, ART-suppressed individuals who had participated in a 2-week ATI.

In this trial, individuals on suppressive ART were randomized to either 40 weeks of continuous ART with a final, open-ended ATI, or a repeated ATIs followed by an open-ended ATI. As there was no difference between the two groups in time-to-viral-rebound or viral setpoint during the open-ended ATI, the full cohort was used for the glycomic analysis. This cohort had a wide distribution of viral rebound times (14-119 days; median=28) and viral setpoints (median=13 675 copies/ml. Johannesburg cohort, serving as a validation cohort—it consisted of 23 HIV-infected, ART-suppressed individuals who had participated in a 2-week ATI; of the 23 participants, 4 did not rebound within the interruption time (data not shown). Glycomic analysis was performed on samples collected immediately before ATI in both cohorts.

B. IgG isolation—Bulk IgG was purified from plasma using Pierce Protein G Spin Plate (Thermo Fisher,Waltham,Massachusetts,USA).

C. N-glycan analysis using capillary electrophoresis

For both plasma and bulk IgG, N-glycans were released using peptide-N-glycosidase F (PNGase F) and labeled with 8-aminopyrene-1,3,6-trisulfonic acid (APTS) using the GlycanAssure APTS Kit (Thermo Fisher), following the manufacturer's protocol. Labeled N-glycans were analyzed using the 3500 Genetic Analyzer capillary electrophoresis system (Thermo Fisher). IgG N-glycan samples were separated into 17 peaks and total plasma N-glycans into 25 peaks (see Giron, L. B., et al. AIDS 34, 681-686, which is incorporated by reference herein). Relative abundance of N-glycan structures was quantified by calculating the area under the curve of each glycan structure divided by the total glycans using the Applied Biosystems GlycanAssure Data Analysis Software Version 2.0 (Thermo Fisher).

D. Glycan analysis using lectin array—IgGs mostly contain N-linked glycans (IgG3 has O-linked glycans), so capillary electrophoresis is mostly sufficient to profile the IgG glycomes. To profile plasma total glycome, we also used the lectin microarray as it enables analysis of multiple glycan structures; it employs a panel of 45 immobilized lectins with known glycan binding specificity (the specific lectins immobilized in the chip and their glycan-binding specificity are in FIG. 19). Plasma proteins were labeled with Cy3 and hybridized to the lectin microarray. The resulting chips were scanned for fluorescence intensity on each lectin-coated spot using an evanescent-field fluorescence scanner GlycoStation Reader (GlycoTechnica Ltd., Yokohama, Kanagawa, Japan), and data were normalized using the global normalization method.

E. Statistical Analysis

Cox proportional-hazards model and log-rank test for time-to-viral-rebound data, two-group t-test or Mann-Whitney test for two-group comparisons, Pearson correlation coefficient (for normally distributed data sets) or Spearman's rank correlation coefficient (for nonnormally distributed data sets) for bivariate correlation analysis, and Wilcoxon signed-rank test for matched-pairs comparison were used for statistical analyses. The Shapiro-Wilk test was used for normality. Statistical analyses were performed in R and Prism 7.0 (GraphPad Software Inc., San Diego, CA, USA).

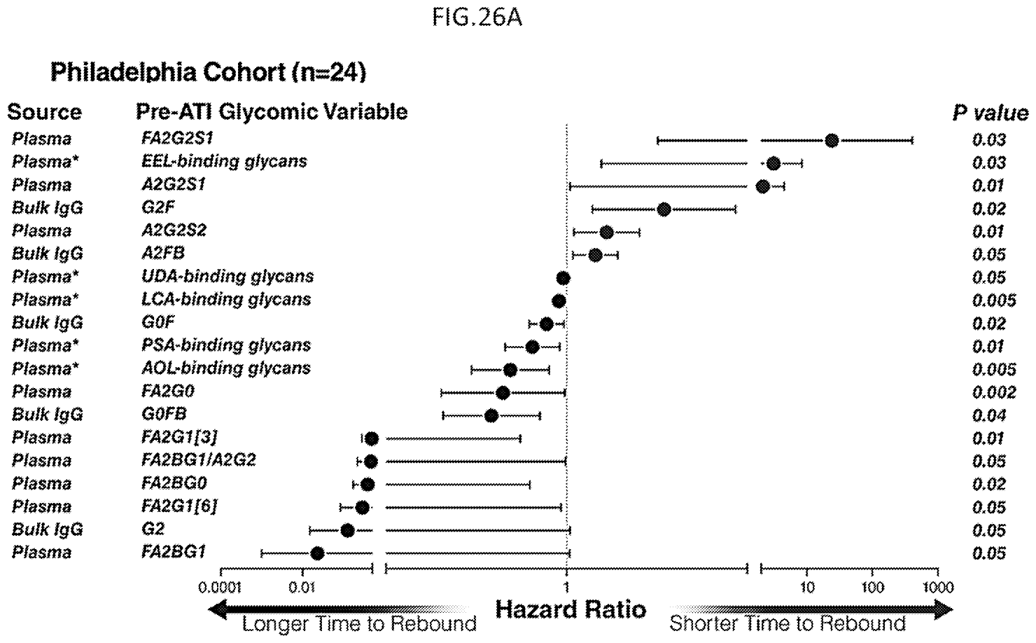

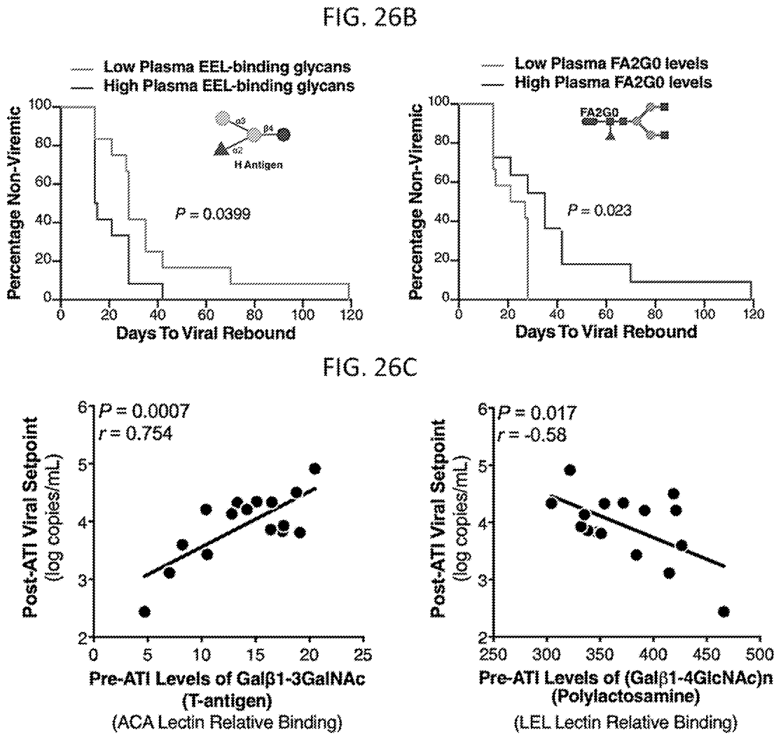

F. Pre-analytic treatment interruption plasma and bulk IgG glycans associate with post-analytic treatment interruption time-to-viral-rebound in the Philadelphia cohort—Using the Cox proportional-hazards model, we found that higher pre-ATI levels of the IgG glycan, G2, were significantly associated with a longer time-to-viral rebound (hazard ratio=0.12, P=0.05) (FIG. 26A). We also observed that G2 glycan levels were affected by viral rebound, being significantly lower at viral rebound (P=0.02) and viral setpoint (P=0.009) than at pre-ATI levels. In addition to G2, we identified several predictive glycomic traits in plasma (FIG. 26A), for example, levels of FA2BG1, a non-sialylated, core-fucosylated glycomic trait, associated with a longer time-to-viral rebound (hazard ratio=0.023, P=0.05), whereas FA2G2S1, a sialylated glycomic trait, associated with a shorter time-to-viral-rebound (hazard ratio=24.1, P=0.028). We also tested these 19 glycomic biomarkers using the Mantel-Cox survival test and the levels of two glycan traits (FA2G0 and EEL-binding glycans) significantly informed time-to-viral-rebound (FIG. 26B). Several other glycan structures trended toward significance, including (FA2G1, G2F, FA2BG0, and A2G2S1).

G. Pre-analytic treatment interruption plasma glycomic signatures associate with post-analytic treatment interruption viral setpoint—Among the pre-ATI plasma glycomic signatures that informed a lower post-ATI viral setpoint was T-antigen (Galb1-3GalNAc) (r=0.75, P=0.0007; FIG. 26C). Only one pre-ATI glycomic signature informed a higher post-ATI viral setpoint, polylactosamine (r=−0.58, P=0.017; FIG. 26C).

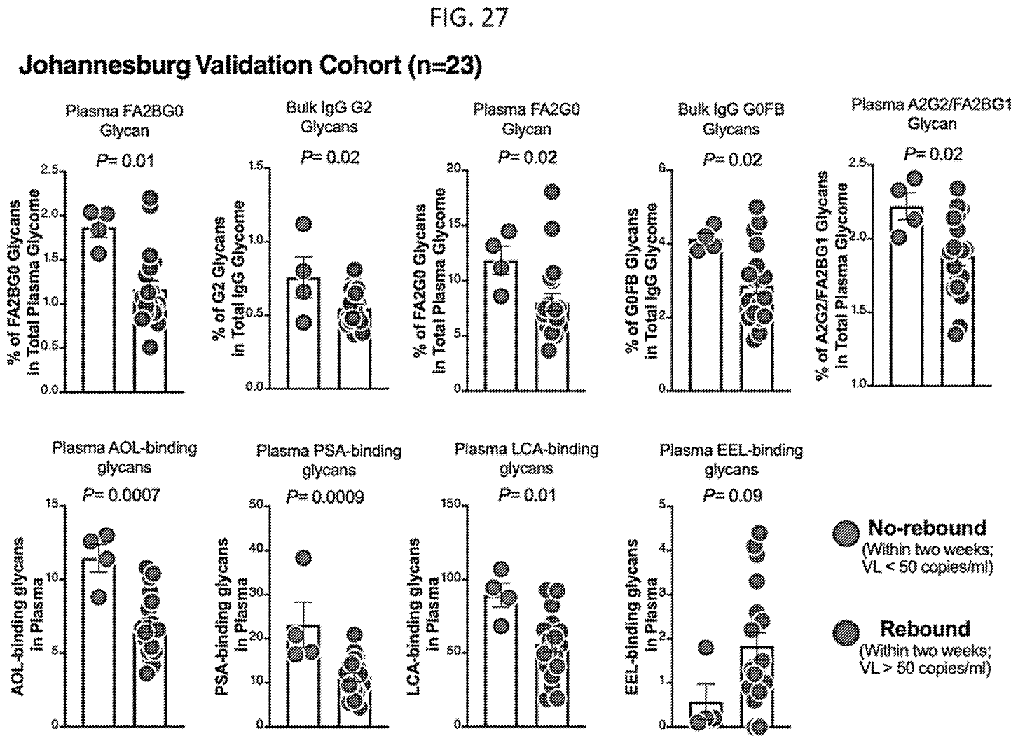

H. Plasma and IgG viral rebound biomarkers are validated in an independent and geographically distinct cohort—To conduct an initial validation of the observations from the Philadelphia cohort, the plasma and bulk IgG glycomic profiles in the Johannesburg cohort, in which 4 out of 23 individuals did not experience viral rebound within a 2-week ATI, were examined. It was found that pre-ATI levels of eight of the glycan signatures that associated with longer time-to-viral-rebound in the Philadelphia cohort (from FIG. 26A) were also higher in the Johannesburg cohort non-rebound group than in the rebound group (P<0.05; FIG. 27). Pre-ATI level of one glycan signature (EEL-binding glycans) that predicted shorter time-to viral-rebound (from FIG. 26A) also trended lower in the non-rebound group compared with the rebound group (FIG. 27).

We identified several novel and non-invasive pre-ATI plasma and bulk IgG glycomic biomarkers that inform time-to-viral-rebound and viral setpoint upon ART cessation. Out of the 19 glycan structures identified in the Philadelphia cohort, nine were initially validated in a geographically distinct cohort, suggesting that these nine glycans (including FA2G0, FA2BG0, G2, fucosylated glycans [AOL, LCA, and PSA-binding glycans, and EEL-binding glycans) are likely the most promising biomarkers. Currently, the only way to evaluate the efficacy of potential HIV curative strategies in clinical trials is by ATI. However, ATIs involve risks to the participants and can lead to viral transmission. Noninvasive, easy-to-measure, pre-ATI biomarkers, such as we have identified here, have the potential to inform outcomes of ATI during HIV curative clinical trials and mitigate some of the risk associated with ATI in those where biomarkers indicate a low likelihood of delayed viral rebound. Plasma and bulk IgG glycomic signatures are easily accessible for clinical monitoring using capillary electrophoresis and lectin-based assays. Specifically, lectin measures can be adapted to a simple ELISA format for fast, high-throughput screening of targeted glycomic signature. This screening could provide information on potential outcomes for participants in HIV curative clinical trials and could also allow targeted recruitment of participants that may be best suited to test HIV curative agents in trials including an ATI.

Plasma glycoproteins enter the circulation from organs through active secretion or leakage, and several studies have shown that glycosylation of such circulating glycoproteins can reflect the inflammatory states of these organs during chronic diseases. Further investigation needs to examine whether the link between plasma glycomic signatures described here and time-to-viral-rebound is a reflection of systemic and/or organ-specific inflammatory status. Independently of inflammation, several studies indicated that circulating glycomes (including on antibodies) can mechanistically regulate immune effector mechanisms. For example, non-fucosylated glycan traits (as G2, which associated with longer time-to-viral-rebound) can mediate higher ADCC. Galactose also has been shown to associate with higher ADCC, CDC and ADCP. However, the role of galactosylation in ADCC activity is controversial. This controversy might be related to the location of the galactose on the glycan structure. During HIV infection, lack of galactosylation was linked to enhanced natural killer cell activity in spontaneous controllers of HIV who control viral replication in the absence of ART. During ART suppression, we recently showed that levels of galactosylated glycans negatively associate with levels of CD4+ T-cell associated HIV DNA and RNA. Whether ART plays a role in this controversy warrants further investigation. Together, the described glycomic signature warrants further examination of its potential steady-state impact on cellular antiviral mechanisms during ART-suppressed HIV infection.

In conclusion, our study provides first-in-class, non-invasive candidate plasma biomarkers, for clinical monitoring on ART that may serve as predictors of time-to-viral-rebound and viral setpoint after treatment interruption to mitigate ATI risks and accelerate the progress toward a cure.

Higher pre-ATI levels of the IgG glycan, G2, were significantly associated with a longer time-to-viral-rebound (hazard ratio=0.12, P=0.05). In addition to G2, we identified several predictive glycomic traits in plasma, for example, levels of FA2BG1, a nonsialylated, core-fucosylated glycan, associated with a longer time-to-viral-rebound (hazard ratio=0.023, P=0.05), whereas FA2G2S1, a sialylated glycan, associated with a shorter time-to-viral-rebound (hazard ratio=24.1, P=0.028). Additionally, pre-ATI plasma glycomic signatures associated with a lower viral setpoint, for example, T-antigen (Galb1-3GalNAc) (r=0.75, P=0.0007), or a higher viral setpoint, for example, polylactosamine (r=0.58, P=0.01). These results were initially validated in the Johannesburg Cohort.

Conclusion: We describe first-in-class, non-invasive, plasma and IgG glycomic biomarkers that inform time-to-viral-rebound and viral setpoint in two geographically distinct cohorts.

### REFERENCES

1 Deeks, S. G. & Barre-Sinoussi, F. Public health: Towards a cure for HIV. Nature 487, 293-294, doi:487293a [pii] 10.1038/487293a (2012), PMID:22810677.
2 Papasavvas, E., et al. Analytical antiretroviral therapy interruption does not irreversibly change preinterruption levels of cellular HIV. Aids 32, 1763-1772, doi:10.1097/QAD.0000000000001909 (2018), PMID:30045057, PMC6289896.
3 Salantes, D. B., et al. HIV-1 latent reservoir size and diversity are stable following brief treatment interruption. J Clin Invest 128, 3102-3115, doi:10.1172/JCI120194 (2018), PMID:29911997, PMC6026010.
4 van Lunzen, J. & Hoffmann, C. Virological rebound and its consequences during treatment interruption. Curr Opin HIV AIDS 2, 1-5, doi:10.1097/COH.0b013e328011aab1 (2007), PMID:19372858.
5 Julg, B., et al. Recommendations for analytical antiretroviral treatment interruptions in HIV research trials-report of a consensus meeting. The lancet. HIV 6, e259-e268, doi:10.1016/S2352-3018(19)30052-9 (2019), PMID:30885693, PMC6688772.
6 Hurst, J., et al. Immunological biomarkers predict HIV-1 viral rebound after treatment interruption. Nature communications 6, 8495, doi:10.1038/ncomms9495 (2015), PMID:26449164, PMC4633715.
7 Williams, J. P., et al. HIV-1 DNA predicts disease progression and post-treatment virological control. Elife 3, e03821, doi:10.7554/eLife.03821 (2014), PMID:25217531, PMC4199415.
8 Li, J. Z., et al. The size of the expressed HIV reservoir predicts timing of viral rebound after treatment interruption. Aids 30, 343-353. doi:10.1097/QAD.0000000000000953 (2016), PMID:26588174, PMC4840470.
9 Pasternak, A. O., et al. Cell-associated HIV-1 RNA predicts viral rebound and disease progression after discontinuation of temporary early ART. JCI Insight 5, doi:10.1172/jci.insight.134196 (2020), PMID:32097124, PMC7213790.
10 Pacanowski, J., et al. Early plasmacytoid dendritic cell changes predict plasma HIV load rebound during primary infection. The Journal of infectious diseases 190, 1889-1892, doi:10.1086/425020 (2004), PMID:15499547.
11 Goujard, C., et al. HIV-1 control after transient antiretroviral treatment initiated in primary infection: role of patient characteristics and effect of therapy. Antivir Ther 17, 1001-1009, doi:10.3851/IMP2273 (2012), PMID:22865544.
12 Lodi, S., et al. Immunovirologic control 24 months after interruption of antiretroviral therapy initiated close to HIV seroconversion. Arch Intern Med 172, 1252-1255, doi:1221717 [pii] 10.1001/archinternmed.2012.2719 (2012), PMID:22826124.
13 Maenza, J., et al. How often does treatment of primary HIV lead to post-treatment control? Antivir Ther 20, 855-863, doi:10.3851/IMP2963 (2015), PMID:25906138.
14 Cockerham, L. R., et al. Post-Treatment Controllers: Role in HIV “Cure” Research. Curr HIV/AIDS Rep 13, 1-9, doi:10.1007/s11904-016-0296-x (2016), PMID:26781112.
15 Martin, G. E. & Frater, J. Post-treatment and spontaneous HIV control. Curr Opin HIV AIDS 13, 402-407, doi:10.1097/COH.0000000000000488 (2018), PMID:29878914,
16 Berkes, E., et al. The analysis of the human plasma N-glycome in endometriosis patients. Eur J Obstet Gynecol Reprod Biol 171, 107-115, doi:10.1016/j.ejogrb.2013.08.008 (2013), PMID:24051301.
17 Hennig, R., et al. Towards personalized diagnostics via longitudinal study of the human plasma N-glycome. Biochim Biophys Acta 1860, 1728-1738, doi:10.1016/j.bbagen.2016.03.035 (2016), PMID:27038647.
18 Keser, T., et al. Increased plasma N-glycome complexity is associated with higher risk of type 2 diabetes. Diabetologia 60, 2352-2360, doi:10.1007/s00125-017-4426-9 (2017), PMID:28905229.
19 Lauc, G., et al. Mechanisms of disease: The human N-glycome. Biochim Biophys Acta 1860, 1574-1582, doi:10.1016/j.bbagen.2015.10.016 (2016), PMID:26500099.
20 Novokmet, M., et al. Changes in IgG and total plasma protein glycomes in acute systemic inflammation. Scientific reports 4, 4347, doi:10.1038/srep04347 (2014), PMID:24614541, PMC3949295.
21 Reiding, K. R., et al. Human Plasma N-glycosylation as Analyzed by Matrix-Assisted Laser Desorption/Ionization-Fourier Transform Ion Cyclotron Resonance-MS Associates with Markers of Inflammation and Metabolic Health. Mol Cell Proteomics 16, 228-242, doi:10.1074/mcp.M116.065250 (2017), PMID:27932526, PMC5294210.
22 Stumpo, K. A. & Reinhold, V. N. The N-glycome of human plasma. J Proteome Res 9, 4823-4830, doi:10.1021/pr100528k (2010), PMID:20690605, PMC2933516.
23 Trbojevic-Akmacic, I., et al. Plasma N-glycome composition associates with chronic low back pain. Biochim Biophys Acta 1862, 2124-2133, doi:10.1016/j.bbagen.2018.07.003 (2018), PMID:29981899.
24 Trbojevic Akmacic, I., et al. Inflammatory bowel disease associates with proinflammatory potential of the immunoglobulin G glycome. Inflamm Bowel Dis 21, 1237-1247, doi:10.1097/MIB.0000000000000372 (2015), PMID:25895110, PMC4450892.
25 Vučković, F., et al. Systemic lupus erythematosus associates with the decreased immunosuppressive potential of the IgG glycome. Arthritis & Rheumatology 67, 2978-2989, doi:10.1002/art.39273 (2015), PMID:26200652, PMC4626261.
26 Vuckovic, F., et al. IgG Glycome in Colorectal Cancer. Clinical cancer research: an official journal of the American Association for Cancer Research 22, 3078-3086, doi:10.1158/1078-0432.CCR-15-1867 (2016), PMID:26831718.
27 Lemmers, R. F. H., et al. IgG glycan patterns are associated with type 2 diabetes in independent European populations. Biochimica et biophysica acta 1861, 2240-2249, doi:10.1016/j.bbagen.2017.06.020 (2017), PMID:28668296.
28 An, H. J. & Lebrilla, C. B. A glycomics approach to the discovery of potential cancer biomarkers. Methods in molecular biology 600, 199-213, doi:10.1007/978-1-60761-454-8_14 (2010), PMID:19882130.
29 Moremen, K. W., et al. Vertebrate protein glycosylation: diversity, synthesis and function. Nature reviews. Molecular cell biology 13, 448-462, doi:10.1038/nrm3383 (2012), PMID:22722607, PMC3934011.
30 Willerson, J. T. & Ridker, P. M. Inflammation as a cardiovascular risk factor. Circulation 109, II2-10, doi:10.1161/01.CIR.0000129535.04194.38 (2004), PMID:15173056.
31 Akinkuolie, A. O., et al. A novel protein glycan biomarker and future cardiovascular disease events. Journal of the American Heart Association 3, e001221, doi:10.1161/JAHA.114.001221 (2014), PMID:25249300, 4323825.
32 Colomb, F., et al. Breaking the Glyco-Code of HIV Persistence and Immunopathogenesis. Curr HIV/AIDS Rep 16, 151-168, doi:10.1007/s11904-019-00433-w (2019), PMID:30707400, PMC6441623.
33 Lu, L. L., et al. Beyond binding: antibody effector functions in infectious diseases. Nature reviews. Immunology 18, 46-61, doi:10.1038/nri.2017.106 (2018), PMID:29063907, PMC6369690.
34 Kaneko, Y., et al. Anti-inflammatory activity of immunoglobulin G resulting from Fc sialylation. Science 313, 670-673, doi:10.1126/science.1129594 (2006), PMID:16888140.
35 Karsten, C. M., et al. Anti-inflammatory activity of IgG1 mediated by Fc galactosylation and association of FcgammaRIIB and dectin-1. Nature medicine 18, 1401-1406, doi:10.1038/nm.2862 (2012), PMID:22922409, PMC3492054.
36 Thomann, M., et al. Fc-galactosylation modulates antibody-dependent cellular cytotoxicity of therapeutic antibodies. Molecular immunology 73, 69-75, doi:10.1016/j.molimm.2016.03.002 (2016), PMID:27058641.
37 Shields, R. L., et al. Lack of fucose on human IgG1 N-linked oligosaccharide improves binding to human Fcgamma RIII and antibody-dependent cellular toxicity. The Journal of biological chemistry 277, 26733-26740, doi:10.1074/jbc.M202069200 (2002), PMID:11986321.
38 Brindle, J. T., et al. Rapid and noninvasive diagnosis of the presence and severity of coronary heart disease using 1H-NMR-based metabonomics. Nat Med 8, 1439-1444, doi:10.1038/nm1202-802 (2002), PMID:12447357.
39 Gabbani, T., et al. Metabolomic analysis with (1)H-NMR for non-invasive diagnosis of hepatic fibrosis degree in patients with chronic hepatitis C. Dig Liver Dis 49, 1338-1344, doi:10.1016/j.dld.2017.05.018 (2017), PMID:28625405.
40 Glinton, K. E. & Elsea, S. H. Untargeted Metabolomics for Autism Spectrum Disorders: Current Status and Future Directions. Front Psychiatry 10, 647, doi:10.3389/fpsyt.2019.00647 (2019), PMID:31551836, PMC6746843.
41 Oeckl, P. & Otto, M. A Review on MS-Based Blood Biomarkers for Alzheimer's Disease. Neurol Ther 8, 113-127, doi:10.1007/s40120-019-00165-4 (2019), PMID:31833028, PMC6908529.
42 Sengupta, N., et al. Analysis of colorectal cancers in British Bangladeshi identifies early onset, frequent mucinous histotype and a high prevalence of RBFOX1 deletion. Mol Cancer 12, 1, doi:10.1186/1476-4598-12-1 (2013), PMID:23286373, PMC3544714.
43 Yang, L., et al. Application of metabolomics in the diagnosis of breast cancer: a systematic review. J Cancer 11, 2540-2551, doi:10.7150/jca.37604 (2020), PMID:32201524, PMC7066003.
44 Zhou, X., et al. Polyunsaturated fatty acids metabolism, purine metabolism and inosine as potential independent diagnostic biomarkers for major depressive disorder in children and adolescents. Mol Psychiatry 24, 1478-1488, doi:10.1038/s41380-018-0047-z (2019), PMID:29679072, PMC6756100.
45 Davaatseren, M., et al. Poly-gamma-glutamic acid attenuates angiogenesis and inflammation in experimental colitis. Mediators Inflamm 2013, 982383, doi:10.1155/2013/982383 (2013), PMID:23766568, PMC3671540.
46 Lee, H. J. & Yang, S. J. Supplementation with Nicotinamide Riboside Reduces Brain Inflammation and Improves Cognitive Function in Diabetic Mice. Int J Mol Sci 20, doi:10.3390/ijms20174196 (2019), PMID:31461911, PMC6747453.
47 Samer, S., et al. Nicotinamide activates latent HIV-1 ex vivo in ART suppressed individuals, revealing higher potency than the association of two methyltransferase inhibitors, chaetocin and BIX01294. Braz J Infect Dis 24, 150-159, doi:10.1016/j.bjid.2020.01.005 (2020), PMID:32105620.
48 Teixeira, G. Q., et al. Anti-inflammatory Chitosan/Poly-gamma-glutamic acid nanoparticles control inflammation while remodeling extracellular matrix in degenerated intervertebral disc. Acta Biomater 42, 168-179, doi:10.1016/j.actbio.2016.06.013 (2016), PMID:27321188.
49 Villeda-Gonzalez, J. D., et al. Nicotinamide reduces inflammation and oxidative stress via the cholinergic system in fructose-induced metabolic syndrome in rats. Life Sci 250, 117585, doi:10.1016/j.lfs.2020.117585 (2020), PMID:32243928.
50 Giron, L. B., et al. Plasma and antibody glycomic biomarkers of time to HIV rebound and viral setpoint. AIDS 34, 681-686, doi:10.1097/QAD.0000000000002476 (2020), PMID:31972605, PMC7072000.
51 Datta, P. K., et al. Glutamate metabolism in HIV-1 infected macrophages: Role of HIV-1 Vpr. Cell cycle 15, 2288-2298, doi:10.1080/15384101.2016.1190054 (2016), PMID:27245560, PMC5004675.
52 Palmer, C. S., et al. Metabolically active CD4+ T cells expressing Glut1 and OX40 preferentially harbor HIV during in vitro infection. FEBS letters 591, 3319-3332, doi:10.1002/1873-3468.12843 (2017), PMID:28892135, PMC5658250.
53 Routy, J. P., et al. Following the elite: Targeting immunometabolism to limit HIV pathogenesis. EBioMedicine 42, 8-9, doi:10.1016/j.ebiom.2019.03.053 (2019), PMID:30910485, PMC6491710.
54 Taylor, H. E., et al. mTOR Overcomes Multiple Metabolic Restrictions to Enable HIV-1 Reverse Transcription and Intracellular Transport. Cell reports 31, 107810, doi:10.1016/j.celrep.2020.107810 (2020), PMID:32579936, PMC7389891.
55 Ushakov, N. A., et al. [Soft contact lenses in the treatment of eye burns of mild and moderate severity]. Oftalmol Zh, 440-441 (1988), PMID:3237378.
56 Valle-Casuso, et al. Cellular Metabolism Is a Major Determinant of HIV-1 Reservoir Seeding in CD4(+) T Cells and Offers an Opportunity to Tackle Infection. Cell Metab 29, 611-626 e615, doi:10.1016/j.cmet.2018.11.015 (2019), PMID:30581119.
57 Papasavvas, E., et al. Randomized, controlled trial of therapy interruption in chronic HIV-1 infection. PLoS medicine 1, e64, doi:10.1371/journal.pmed.0010064 (2004), PMID:15630469, PMC539050.
58 Volberding, P., et al. Antiretroviral therapy in acute and recent HIV infection: a prospective multicenter stratified trial of intentionally interrupted treatment. Aids 23, 1987-1995, doi:10.1097/QAD.0b013e32832eb285 (2009), PMID:19696651, PMC2888600.
59 Kilby, J. M., et al. A randomized, partially blinded phase 2 trial of antiretroviral therapy, HIV-specific immunizations, and interleukin-2 cycles to promote efficient control of viral replication (ACTG A5024). The Journal of infectious diseases 194, 1672-1676, doi:10.1086/509508 (2006), PMID:17109338.
60 Jacobson, J. M., et al. Evidence that intermittent structured treatment interruption, but not immunization with ALVAC-HIV vCP1452, promotes host control of HIV replication: the results of AIDS Clinical Trials Group 5068. The Journal of infectious diseases 194, 623-632, doi:10.1086/506364 (2006), PMID:16897661.
61 Skiest, D. J., et al. Interruption of antiretroviral treatment in HIV-infected patients with preserved immune function is associated with a low rate of clinical progression: a prospective study by AIDS Clinical Trials Group 5170. The Journal of infectious diseases 195, 1426-1436, doi:10.1086/512681 (2007), PMID:17436222.
62 Rosenberg, E. S., et al. Safety and immunogenicity of therapeutic DNA vaccination in individuals treated with antiretroviral therapy during acute/early HIV-1 infection. PloS one 5, e10555, doi:10.1371/journal.pone.0010555 (2010), PMID:20479938, PMC2866663.
63 Schooley, R. T., et al. AIDS clinical trials group 5197: a placebo-controlled trial of immunization of HIV-1-infected persons with a replication-deficient adenovirus type 5 vaccine expressing the HIV-1 core protein. The Journal of infectious diseases 202, 705-716, doi:10.1086/655468 (2010), PMID:20662716, PMC2916952.
64 Etemad, B., et al. Learning From the Exceptions: HIV Remission in Post-treatment Controllers. Frontiers in immunology 10, 1749, doi:10.3389/fimmu.2019.01749 (2019), PMID:31396237, PMC6668499.
65 Namazi, G., et al. The Control of HIV After Antiretroviral Medication Pause (CHAMP) Study: Posttreatment Controllers Identified From 14 Clinical Studies. The Journal of infectious diseases 218, 1954-1963, doi:10.1093/infdis/jiy479 (2018), PMID:30085241, PMC6217727.
66 Sharaf, R., et al. HIV-1 proviral landscapes distinguish posttreatment controllers from noncontrollers. The Journal of clinical investigation 128, 4074-4085, doi:10.1172/JC1120549 (2018), PMID:30024859, PMC6118642.
67 Kornberg, M. D., et al. Dimethyl fumarate targets GAPDH and aerobic glycolysis to modulate immunity. Science 360, 449-453, doi:10.1126/science.aan4665 (2018), PMID:29599194, PMC5924419.
68 Palsson-McDermott, E. M. & O'Neill, L. A. J. Targeting immunometabolism as an anti-inflammatory strategy. Cell research 30, 300-314, doi:10.1038/s41422-020-0291-z (2020), PMID:32132672, PMC7118080.
69 Duette, G., et al. Induction of HIF-lalpha by HIV-1 Infection in CD4(+) T Cells Promotes Viral Replication and Drives Extracellular Vesicle-Mediated Inflammation. mBio 9, doi:10.1128/mBio.00757-18 (2018), PMID:30206166, PMC6134101.
70 Palsson-McDermott, E. M., et al. Pyruvate kinase M2 regulates Hif-lalpha activity and IL-1beta induction and is a critical determinant of the warburg effect in LPS-activated macrophages. Cell Metab 21, 65-80, doi:10.1016/j.cmet.2014.12.005 (2015), PMID:25565206, PMC5198835.
71 Peng, M., et al. Aerobic glycolysis promotes T helper 1 cell differentiation through an epigenetic mechanism. Science 354, 481-484, doi:10.1126/science.aaf6284 (2016), PMID:27708054, PMC5539971.
72 Chavez, L., et al. In vivo, in vitro, and in silico analysis of methylation of the HIV-1 provirus. Methods 53, 47-53, doi:10.1016/j.ymeth.2010.05.009 (2011), PMID:20670606, 3566233.
73 Jordan, A., et al. HIV reproducibly establishes a latent infection after acute infection of T cells in vitro. EMBO J 22, 1868-1877, doi:10.1093/emboj/cdgl88 (2003), PMID:12682019, PMC154479.
74 Abdel-Mohsen, M., et al. Human Galectin-9 Is a Potent Mediator of HIV Transcription and Reactivation. PLoS Pathog 12, e1005677, doi:10.1371/journal.ppat.1005677 (2016), PMID:27253379, PMC4890776.
75 Colomb, F., et al. Galectin-9 Mediates HIV Transcription by Inducing TCR-Dependent ERK Signaling. Front Immunol 10, 267, doi:10.3389/fimmu.2019.00267 (2019), PMID:30842775, PMC6391929.
76 Tateno, H., et al.. A versatile technology for cellular glycomics using lectin microarray. Methods in enzymology 478, 181-195, doi:10.1016/S0076-6879(10)78008-3 (2010), PMID:20816480.
77 Tateno, H., et al. Glycome diagnosis of human induced pluripotent stem cells using lectin microarray. The Journal of biological chemistry 286, 20345-20353, doi:10.1074/jbc.M111.231274 (2011), PMID:21471226, 3121447.
78 Hirabayashi, J. et al. Development and Applications of the Lectin Microarray. Topics in current chemistry 367, 105-124, doi:10.1007/128_2014_612 (2015), PMID:25821171.
79 Abdel-Mohsen, M., et al. Recommendations for measuring HIV reservoir size in cure-directed clinical trials. Nature medicine 26, 1339-1350, doi:10.1038/s41591-020-1022-1 (2020), PMID:32895573.
80 Bruner, K. M., et al. A quantitative approach for measuring the reservoir of latent HIV-1 proviruses. Nature 566, 120-125, doi:10.1038/s41586-019-0898-8 (2019), PMID:30700913, PMC6447073.
81 Jiang, C., et al. Distinct viral reservoirs in individuals with spontaneous control of HIV-1. Nature 585, 261-267, doi:10.1038/s41586-020-2651-8 (2020), PMID:32848246.
82 Peluso, M. J., et al. Differential decay of intact and defective proviral DNA in HIV-1-infected individuals on suppressive antiretroviral therapy. JCI Insight 5, doi:10.1172/jci.insight.132997 (2020), PMID:32045386, PMC7101154.
83 Simonetti, F. R., et al. Intact proviral DNA assay analysis of large cohorts of people with HIV provides a benchmark for the frequency and composition of persistent proviral DNA. Proceedings of the National Academy of Sciences of the United States of America 117, 18692-18700, doi:10.1073/pnas.2006816117 (2020), PMID:32690683, PMC7414172.
84 Kwon, K. J., et al. Different human resting memory CD4(+) T cell subsets show similar low inducibility of latent HIV-1 proviruses. Sci Transl Med 12, doi:10.1126/scitranslmed.aax6795 (2020), PMID:31996465.
85 Lewinski, M. K., et al. Genome-wide analysis of chromosomal features repressing human immunodeficiency virus transcription. J Virol 79, 6610-6619, doi:10.1128/JVI.79.11.6610-6619.2005 (2005), PMID:15890899, PMC1112149.
86 Sherrill-Mix, S., et al. HIV latency and integration site placement in five cell-based models. Retrovirology 10, 90, doi:10.1186/1742-4690-10-90 (2013), PMID:23953889, PMC3765678.
87 Schroder, A. R., et al. HIV-1 integration in the human genome favors active genes and local hotspots. Cell 110, 521-529, doi:10.1016/s0092-8674(02)00864-4 (2002), PMID:12202041.
88 Wang, G. P., et al. HIV integration site selection: analysis by massively parallel pyrosequencing reveals association with epigenetic modifications. Genome Res 17, 1186-1194, doi:10.1101/gr.6286907 (2007), PMID:17545577, PMC1933515.
89 Einkauf, K. B., et al. Intact HIV-1 proviruses accumulate at distinct chromosomal positions during prolonged antiretroviral therapy. J Clin Invest 129, 988-998, doi:10.1172/JCI124291 (2019), PMID:30688658, PMC6391088.
90 Cohn, L. B., et al. HIV-1 integration landscape during latent and active infection. Cell 160, 420-432, doi:10.1016/j.cell.2015.01.020 (2015), PMID:25635456, PMC4371550.
91 Patro, S. C., et al. Combined HIV-1 sequence and integration site analysis informs viral dynamics and allows reconstruction of replicating viral ancestors. Proc Natl Acad Sci USA 116, 25891-25899, doi:10.1073/pnas.1910334116 (2019), PMID:31776247, PMC6925994.
92 Cesana, D et al. HIV-1-mediated insertional activation of STAT5B and BACH2 trigger viral reservoir in T regulatory cells. Nat Commun 8, 498, doi:10.1038/s41467-017-00609-1 (2017), PMID:28887441, PMC5591266.
93 Shehata, H. M., et al. Sugar or Fat?-Metabolic Requirements for Immunity to Viral Infections. Frontiers in immunology 8, 1311, doi:10.3389/fimmu.2017.01311 (2017), PMID:29085369, PMC5649203.
94 Zhang, D., et al. Metabolic regulation of gene expression by histone lactylation. Nature 574, 575-580, doi:10.1038/s41586-019-1678-1 (2019), PMID:31645732, PMC6818755.
95 Freemerman, A. J., et al. Metabolic reprogramming of macrophages: glucose transporter 1 (GLUT1)-mediated glucose metabolism drives a proinflammatory phenotype. The Journal of biological chemistry 289, 7884-7896. doi:10.1074/jbc.M113.522037 (2014), PMID:24492615, PMC3953299.
96 Barrero, C. A., et al. HIV-1 Vpr modulates macrophage metabolic pathways: a SILAC-based quantitative analysis. PloS one 8, e68376, doi:10.1371/journal.pone.0068376 (2013), PMID:23874603, PMC3709966.
97 Tarancon-Diez, L., et al. Immunometabolism is a key factor for the persistent spontaneous elite control of HIV-1 infection. EBioMedicine 42, 86-96, doi:10.1016/j.ebiom.2019.03.004 (2019), PMID:30879922, PMC6491381.
98 Aounallah, M., et al. Current topics in HIV pathogenesis, part 2: Inflammation drives a Warburg-like effect on the metabolism of HIV-infected subjects. Cytokine Growth Factor Rev 28, 1-10, doi:10.1016/j.cytogfr.2016.01.001 (2016), PMID:26851985,
99 Hammoud, D. A., et al. Increased Metabolic Activity on 18F-Fluorodeoxyglucose Positron Emission Tomography-Computed Tomography in Human Immunodeficiency Virus-Associated Immune Reconstitution Inflammatory Syndrome. Clinical infectious diseases: an official publication of the Infectious Diseases Society of America 68, 229-238, doi:10.1093/cid/ciy454 (2019), PMID:30215671, PMC6321853.
100 Saez-Cirion, A. & Sereti, I. Immunometabolism and HIV-1 pathogenesis: food for thought. Nature reviews. Immunology, doi:10.1038/s41577-020-0381-7 (2020), PMID:32764670.
101 Arpaia, N., et al. Metabolites produced by commensal bacteria promote peripheral regulatory T-cell generation. Nature 504, 451-455, doi:10.1038/nature12726 (2013), PMID:24226773, PMC3869884.
102 Dodd, D., et al. A gut bacterial pathway metabolizes aromatic amino acids into nine circulating metabolites. Nature 551, 648-652, doi:10.1038/nature24661 (2017), PMID:29168502, PMC5850949.
103 Chen, J., et al. Plasma Indoleamine 2,3-Dioxygenase Activity Is Associated With the Size of the Human Immunodeficiency Virus Reservoir in Patients Receiving Antiretroviral Therapy. Clinical infectious diseases: an official publication of the Infectious Diseases Society of America 68, 1274-1281, doi:10.1093/cid/ciy676 (2019), PMID:30107503, PMC6451994.
104 Hunt, P. W., et al. Gut epithelial barrier dysfunction and innate immune activation predict mortality in treated HIV infection. The Journal of infectious diseases 210, 1228-1238, doi:10.1093/infdis/jiu238 (2014), PMID:24755434, PMC4192038.
105 Vujkovic-Cvijin, I., et al. HIV-associated gut dysbiosis is independent of sexual practice and correlates with noncommunicable diseases. Nature communications 11, 2448, doi:10.1038/s41467-020-16222-8 (2020), PMID:32415070, PMC7228978.
106 Vujkovic-Cvijin, I., et al. Dysbiosis of the gut microbiota is associated with HIV disease progression and tryptophan catabolism. Sci Transl Med 5, 193ra191, doi:10.1126/scitranslmed.3006438 (2013), PMID:23843452, PMC4094294.
107 Favre, D., et al. Tryptophan catabolism by indoleamine 2,3-dioxygenase 1 alters the balance of TH17 to regulatory T cells in HIV disease. Sci Transl Med 2, 32ra36, doi:10.1126/scitranslmed.3000632 (2010), PMID:20484731, PMC3034445.
108 Masuda, K., et al. Enhanced binding affinity for FcgammaRIIIa of fucose-negative antibody is sufficient to induce maximal antibody-dependent cellular cytotoxicity. Molecular immunology 44, 3122-3131, doi:10.1016/j.molimm.2007.02.005 (2007), PMID:17379311.
109 Naso, M. F., et al. Engineering host cell lines to reduce terminal sialylation of secreted antibodies. MAbs 2, 519-527, doi:10.4161/mabs.2.5.13078 (2010), PMID:20716959, PMC2958573.
110 Raju, T. S. Terminal sugars of Fc glycans influence antibody effector functions of IgGs. Current opinion in immunology 20, 471-478, doi:10.1016/j.coi.2008.06.007 (2008), PMID:18606225.
111 Raju, T. S. & Scallon, B. Fc glycans terminated with N-acetylglucosamine residues increase antibody resistance to papain. Biotechnol Prog 23, 964-971, doi:10.1021/bp070118k (2007), PMID:17571902.
112 Azuma, K., et al. Suppressive effects of N-acetyl-D-glucosamine on rheumatoid arthritis mouse models. Inflammation 35, 1462-1465, doi:10.1007/s10753-012-9459-0 (2012), PMID:22434264.
113 Kubomura, D., et al. Effect of N-acetylglucosamine administration on cartilage metabolism and safety in healthy subjects without symptoms of arthritis: A case report. Exp Ther Med 13, 1614-1621, doi:10.3892/etm.2017.4140 (2017), PMID:28413518, PMC5377572.
114 Salvatore, S., et al. A pilot study of N-acetyl glucosamine, a nutritional substrate for glycosaminoglycan synthesis, in paediatric chronic inflammatory bowel disease. Aliment Pharmacol Ther 14, 1567-1579, doi:10.1046/j.1365-2036.2000.00883.x (2000), PMID:11121904.
115 Konopka, J. B. N-acetylglucosamine (GlcNAc) functions in cell signaling. Scientifica (Cairo) 2012, doi:10.6064/2012/489208 (2012), PMID:23350039, PMC3551598.
116 Mendez-Huergo, S. P., et al., G. A. Galectins: emerging regulatory checkpoints linking tumor immunity and angiogenesis. Curr Opin Immunol 45, 8-15, doi:10.1016/j.coi. 2016.12.003 (2017), PMID:28088061.
117 Smith, L. K., et al. Interleukin-10 Directly Inhibits CD8(+) T Cell Function by Enhancing N-Glycan Branching to Decrease Antigen Sensitivity. Immunity 48, 299-312 e295, doi:10.1016/j.immuni.2018.01.006 (2018), PMID:29396160, PMC5935130.
118 Byrne, B., et al. Sialic acids: carbohydrate moieties that influence the biological and physical properties of biopharmaceutical proteins and living cells. Drug discovery today 12, 319-326, doi:10.1016/j.drudis.2007.02.010 (2007), PMID:17395092.
119 Rabinovich, G. A., et al. Role of galectins in inflammatory and immunomodulatory processes. Biochimica et biophysica acta 1572, 274-284 (2002), PMID:12223275.
120 Zhuo, Y. & Bellis, S. L. Emerging role of alpha2,6-sialic acid as a negative regulator of galectin binding and function. The Journal of biological chemistry 286, 5935-5941, doi:10.1074/jbc.R110.191429 (2011), PMID:21173156, PMC3057866.
121 Ley, K. The role of selectins in inflammation and disease. Trends Mol Med 9, 263-268 (2003), PMID:12829015.
122 Campbell, B. J., et al. Direct demonstration of increased expression of Thomsen-Friedenreich (TF) antigen in colonic adenocarcinoma and ulcerative colitis mucin and its concealment in normal mucin. The Journal of clinical investigation 95, 571-576, doi:10.1172/JCI117700 (1995), PMID:7860740, PMC295516.
123 Chia, J., et al. Short O-GalNAc glycans: regulation and role in tumor development and clinical perspectives. Biochimica et biophysica acta 1860, 1623-1639, doi:10.1016/j.bbagen.2016.03.008 (2016), PMID:26968459.
124 Howard, D. R. & Taylor, C. R. An antitumor antibody in normal human serum: reaction of anti-T with breast carcinoma cells. Oncology 37, 142-148, doi:10.1159/000225423 (1980), PMID:6244524.
125 Patil, S. A., et al. Overexpression of alpha2,3sialyl T-antigen in breast cancer determined by miniaturized glycosyltransferase assays and confirmed using tissue microarray immunohistochemical analysis. Glycoconjugate journal 31, 509-521, doi:10.1007/s10719-014-9548-4 (2014), PMID:25142811, PMC4323378.
126 Pearce, O. M. T. Cancer glycan epitopes: biosynthesis, structure and function. Glycobiology 28, 670-696, doi:10.1093/glycob/cwy023 (2018), PMID:29546349,
127 Springer, G. F. T and Tn, general carcinoma autoantigens. Science 224, 1198-1206, doi:10.1126/science.6729450 (1984), PMID:6729450.
128 Springer, G. F., et al. Blood group MN antigens and precursors in normal and malignant human breast glandular tissue. Journal of the National Cancer Institute 54, 335-339 (1975), PMID:163330.
129 Stowell, S. R., et al. Protein glycosylation in cancer. Annual review of pathology 10, 473-510, doi:10.1146/annurev-pathol-012414-040438 (2015), PMID:25621663, PMC4396820.
130 van Vliet, S. J., et al. Regulation of effector T cells by antigen-presenting cells via interaction of the C-type lectin MGL with CD45. Nature immunology 7, 1200-1208, doi:10.1038/ni1390 (2006), PMID:1699849.
131 Bruner, K. M., et al. Defective proviruses rapidly accumulate during acute HIV-1 infection. Nature medicine 22, 1043-1049, doi:10.1038/nm.4156 (2016), PMID:27500724, PMC5014606.
132 Ho, Y. C., et al. Replication-competent noninduced proviruses in the latent reservoir increase barrier to HIV-1 cure. Cell 155, 540-551, doi:10.1016/j.cell.2013.09.020 (2013), PMID:24243014, PMC3896327.
133 Li, J., et al. The mitophagy effector FUNDCl controls mitochondrial reprogramming and cellular plasticity in cancer cells. Science signaling 13, doi:10.1126/scisignal. aaz8240 (2020), PMID:32723812, PMC7484983.
134 Hirabayashi, J., et al. Lectin microarrays: concept, principle and applications. Chemical Society reviews 42, 4443-4458, doi:10.1039/c3cs35419a (2013), PMID:23443201,
135 Tateno, H., et al. A novel strategy for mammalian cell surface glycome profiling using lectin microarray. Glycobiology 17, 1138-1146, doi:10.1093/glycob/cwm084 (2007), PMID: 17693441,
136 Tateno, H., et al. Comparative analysis of core-fucose-binding lectins from Lens culinaris and Pisum sativum using frontal affinity chromatography. Glycobiology 19, 527-536, doi:10.1093/glycob/cwp016 (2009), PMID:19218400,
137 Nagahara, K., et al. Galectin-9 increases Tim-3+ dendritic cells and CD8+ T cells and enhances antitumor immunity via galectin-9-Tim-3 interactions. J Immunol 181, 7660-7669 (2008), PMID:19017954,
138 Uchiyama, N., et al. Optimization of evanescent-field fluorescence-assisted lectin microarray for high-sensitivity detection of monovalent oligosaccharides and glycoproteins. Proteomics 8, 3042-3050, doi:10.1002/pmic.200701114 (2008), PMID:18615430

Each and every patent, patent application, and publication, including websites cited throughout specification are incorporated herein by reference. U.S. Provisional Patent Application No. 63/112,047, filed Nov. 10, 2020, is incorporated herein by reference. While the invention has been described with reference to particular embodiments, it will be appreciated that modifications can be made without departing from the spirit of the invention. Such modifications are intended to fall within the scope of the appended claims.

1. A diagnostic kit for the non-invasive prediction of time-to-viral-rebound in an HIV subject after antiretroviral therapy (ART) interruption comprising reagents that detect and measure the metabolites and glycans comprising:
(a) Plasma A3G3S3 glycans, Pyruvic acid, Plasma T-antigen that binds the lectic MPA, Plasma T-antigen that binds the lectin ACA, Total fucose, L-glutamic acid, and (GlcNAc)n; or
(b) IgG A2 glycans, Plasma A3G3S3 glycans, Total Fucose, Core fucose, Plasma T-antigen that binds lectin ABA, (GlcNAc)n, and L-glutamic acid.

2. (canceled)

3. The kit according to claim 1, wherein each reagent detects and measures a single of said metabolites and glycans.

4. The kit according to claim 1, wherein each reagent is a lectin, optionally associated with a detectable label.

5. The kit according to claim 1, wherein each reagent is an antibody, optionally associated with a detectable label.

6. The kit according to claim 1, further comprising components necessary to perform a protein binding assay.

7. The kit according to claim 1, further comprising components necessary to perform a lectin micro array, a lectin ELISA, lectin blotting, or lectin hybridization.

8. The kit according to claim 1, further comprising components necessary to perform capillary electrophoresis or mass spectrometry.

9. A method for predicting time-to-viral rebound in an HIV-infected subject's sample, comprising detecting and measuring the levels of the combination of metabolites and glycans comprising:
(a) Plasma A3G3S3 glycans, Pyruvic acid, Plasma T-antigen that binds the lectic MPA, Plasma T-antigen that binds the lectin ACA, Total fucose, L-glutamic acid, and (GlcNAc)n; or
(b) IgG A2 glycans, Plasma A3G3S3 glycans, Total Fucose, Core fucose, Plasma T-antigen that binds lectin ABA, (GlcNAc)n, and L-glutamic acid.

10. The method according to claim 9, which predicts time-to-viral rebound by 74-76%.

11. (canceled)

12. The method according to claim 9, which predicts the probability of viral rebound with a sensitivity of 97.5%.

13. The method according to claim 9, which employs the kit of claim 1.

14. The method according to claim 9, which employs the steps of a protein binding assay.

15. The method according to claim 14, which employs the steps of a lectin micro array, a lectin ELISA, lectin blotting, or lectin hybridization.

16. The method according to claim 9, which comprises capillary electrophoresis.

17. The method according to claim 9, which comprises mass spectrometry.

18. A method for treating an HIV-infected subject comprising increasing the in vivo level or expression of one of more of the metabolites and glycans selected from:
(a) Glychocholic acid, Taurochenodeoxycholic acid, Glycoursodeoxycholic acid, D Glucose, Ethylmalonic acid, N-Acetyl glutamic acid, Malonic acid, Ethylmalonic acid, 1, 5, Anhydro D glucitol, Kojic acid, D-ribono-1,4-lactone, Gamma aminobutyric acid GABA, Oxoglutaric acid (α-ketoglutaric acid) and L-glutamic acid, or
(b) Pyruvic acid, L-Lactic acid, Taurine, Glycerol-3-phosphate, Indole-3 lactic acid, Indole 3 pyruvic acid, Imidazole lactic acid, Glycerophospho-N-palmitoyl ethanolamine, 2-oxindole, 3-Indoxyl sulphate, Nicotinamide, and Trimethylamine N-oxide.

19. (canceled)
